(19) United States
(12) Patent Application Publication
(10) Pub. No.: US 2016/0083788 A1
(43) Pub. Date: Mar. 24, 2016

(54) METHOD FOR TARGETED SEQUENCING

(71) Applicant: KEYGENE N.V., Wageningen (NL)

(72) Inventor: René Cornelis Josephus HOGERS, Wageningen (NL)

(73) Assignee: Keygene N.V., Wageningen, (NL)

(21) Appl. No.: 14/677,811

(22) PCT Filed: Jun. 6, 2014
(86) PCT No.: PCT/NL2014/050369

§ 371 Date: Apr. 2, 2015

(30) Foreign Application Priority Data
Jun. 7, 2013 (NL) 2010933

(51) Int. Cl. C12Q 1/68

(52) U.S. Cl. CPC C12Q 1/6874

(57) ABSTRACT

The method of the present invention now provides a technique for generating sequence information from nucleic acid samples based on knowledge from part(s) of the nucleotide sequence. The knowledge of the partial sequence may include knowledge about the presence of restriction sites. The knowledge of the partial sequence can be used to generate adaptor-ligated or nucleotide-elongated fragments. From the combination of information on the ligated adaptor and the Known Nucleotide Sequence Section, probes can be designed. The probes can be used in the provision of circularized fragments that can be sequenced. Combining the known and determined sequences adds sequence information to the already existing sequence information and complements the available genomic sequence information.

### CROSS-REFERENCE TO RELATED APPLICATIONS

This application is a continuation of International Patent Application No. PCT/NL2014/050369, filed Jun. 6, 2014, published as WO 2014/196863, which claims priority to Netherlands Application No. 2010933, filed Jun. 7, 2013, both of which are herein incorporated by reference in their entireties.

### TECHNICAL FIELD OF THE INVENTION

The present invention pertains to the field of determining the nucleotide sequence of nucleic acid samples. More in particular the invention relates to generating further sequence information from nucleic acid samples of which some sequence information is already available.

### BACKGROUND ART

Over the last years, high throughput sequencing methods have become widely available. These methods generate large amounts of sequence data, often in the form of shorter or longer nucleotide sequence fragments (aka reads). The challenge is to assemble these data into draft genome sequences or contigs and to fill the gaps between the fragments in order to come to complete genomes.

WO200511236 describes a method for the amplification of a plurality of target sequences whereby fragments are provided, for instance by using restriction enzymes. The double stranded fragments are denatured to single stranded fragments. To the single stranded fragments, specific double stranded selectors are ligated that may contain primer binding sites and the selector-ligated fragment is circularised. The resulting circular DNA can be amplified and sequenced.

WO2012003374 describes a sequencing method wherein restriction-enzyme digested DNA is circularised via an oligonucleotide set that is complementary to both sides of the fragment. The oligonucleotide set contains a splint oligonucleotide and a vector oligonucleotide. The vector oligonucleotide is ligated between the ends of the fragment and the splint oligonucleotide is complementary to the ends of the fragment and the vector oligonucleotide. The oligonucleotide set can comprise a primer binding site. After removal of the splint oligonucleotide, the circularised fragment can be amplified and sequenced. WO2012003374 requires double stranded constructs prior to ligation.

WO2011067378 describes a method for the amplification of circularised target fragments wherein fragments are generated comprising the target sequence and two complementary probe portions, one of which is located at the end of the target fragment. To the complementary probe portions, double stranded probes are annealed and ligated. The probe-ligated fragments are isolated by using a probe with a immobilisation moiety such as biotin. The fragments can be analysed using sequencing. WO2011067378 requires knowledge of at least two parts of the sequence in order to design a useful probe for the circularization.

WO2008153492 describes a method for introducing sequence elements in a target nucleic acid using a combination of multiple probes.

Prior art uses multiple probes or requires knowledge of multiple parts of the nucleotide sequence of the sample nucleic acid. When for instance restriction fragments are used, the prior art methods use the two known genomics sequences ends of the restriction fragments. There remains a need in the art for methods that provide additional sequence information based on a limited amount of initial sequence information. The present inventors now provide simplified methods that rely on single sequence information that may be located at or near the end of a restriction fragment together with a generic known sequence (adaptor) and uses only one probe to generate circularised nucleic acids that can be amplified and sequenced.

### SUMMARY OF INVENTION

The method of the present invention now provides a technique for generating sequence information from nucleic acid samples based on knowledge from part(s) of the nucleotide sequence. The knowledge of the partial sequence may include knowledge about the presence of restriction sites, which includes knowledge on the statistical occurrence of the presence of restriction sites. The knowledge of the partial sequence can be used to generate adaptor-ligated or nucleotide-elongated fragments. From the combination of information on the ligated adaptor and part of the nucleotide sequence, such as the restriction sites, probes can be designed. The probes can be used in the provision of circularised fragments that can be sequenced. Combining the known and determined sequences adds sequence information to the already existing sequence information and complements the genome sequence.

Thus the invention provides, in one embodiment, a method for obtaining sequence information from a nucleic acid sample, the method comprising the steps of:
a) providing a nucleic acid sample wherein at least part of the nucleotide sequence information for the nucleic acid sample is available in the form of at least one Known Nucleotide Sequence Section;
b) fragmenting the nucleic acid sample to obtain one or more fragments;
c) optionally, blunting the ends of the fragments(s);
d) optionally, adding one or more 3′ nucleotides to the fragments;
e) ligating one or more adaptor(s) to one or both of the ends of the fragment(s) to obtain (an) adaptor-ligated fragment(s);
f) denaturing the adaptor-ligated fragment(s) to obtain (a) denatured adaptor-ligated fragment(s);
g) providing for at least one, preferably for each, optionally selected Known

Nucleotide Sequence Section-containing, denatured adaptor-ligated fragment a circularization probe that comprises at least part of the Known Nucleotide Sequence Section and at least part of the sequence of the adaptor;
h) combining the denatured adaptor-ligated fragment(s) with the circularization probe(s);
i) allowing the circularization probe(s) and the denatured adaptor-ligated fragment(s) to hybridize and form (a) circularized denatured adaptor-ligated fragment(s);
j) optionally, removing an overhang;

k) optionally, filling in missing nucleotides between (part of) the Known Nucleotide Sequence Section and (part of) the adaptor;
l) ligating the ends of the circularized adaptor-ligated fragment(s) to obtain (a) ligated circularized adaptor-ligated fragment(s); and
m) sequencing the ligated circularized adaptor-ligated fragment(s); wherein, for each fragment, sequence information of only one single Known Nucleotide Sequence section is required to obtain sequence information of the ligated circularized adaptor-ligated fragment(s).

The invention also provides, in one embodiment, a method for obtaining sequence information from a nucleic acid sample, the method comprising the steps of:
a) providing a nucleic acid sample wherein at least part of the nucleotide sequence information for the nucleic acid sample is available in the form of at least one Known Nucleotide Sequence Section;
b) fragmenting the nucleic acid sample to obtain one or more fragments;
c) optionally, blunting the ends of the fragments(s);
d) optionally, adding one or more 3′ nucleotides to the fragments;
e) ligating one or more adaptor(s) to one or both of the ends of the fragment(s) to obtain (an) adaptor-ligated fragment(s);
f) providing for at least one, preferably for each, optionally selected Known Nucleotide Sequence Section-containing, adaptor-ligated fragment a circularization probe that comprises at least part of the Known Nucleotide Sequence Section and at least part of the sequence of the adaptor;
g) combining the adaptor-ligated fragment(s) with the circularization probe(s);
h) denaturing the adaptor-ligated fragment(s) to obtain (a) denatured adaptor-ligated fragment(s);
i) allowing the circularization probe(s) and the denatured adaptor-ligated fragment(s) to hybridize and form (a) circularized denatured adaptor-ligated fragment(s);
j) optionally, removing an overhang;
k) optionally, filling in missing nucleotides between (part of) the Known Nucleotide Sequence Section and (part of) the adaptor;
l) ligating the ends of the circularized adaptor-ligated fragment(s) to obtain (a) ligated circularized adaptor-ligated fragment(s); and
m) sequencing the ligated circularized adaptor-ligated fragment(s); wherein, for each fragment, sequence information of only one single Known Nucleotide Sequence section is required to obtain sequence information of the ligated circularized adaptor-ligated fragment(s).

In another embodiment, a method is provided for obtaining sequence information from a nucleic acid sample, the method comprising the steps of:
a) providing a nucleic acid sample wherein at least part of the nucleotide sequence information for the nucleic acid sample is available in the form of at least one Known Nucleotide Sequence Section;
b) fragmenting the nucleic acid sample to obtain one or more fragments;
c) optionally, blunting the ends of the fragments(s);
d) adding one or more 3′ nucleotides, preferably 10 to 20 nucleotides to the fragment(s) to obtain (a) nucleotide-elongated fragment(s);
e) denaturing the nucleotide-elongated fragment(s) to obtain (a) denatured nucleotide-elongated fragment(s);
f) providing for at least one, preferably for each, optionally selected Known Nucleotide Sequence Section-containing, denatured nucleotide-elongated fragment a circularization probe that comprises at least part of the Known Nucleotide Sequence Section and at least part of the sequence of the nucleotide-elongated sequence;
g) combining the denatured nucleotide-elongated fragment(s) with the circularization probe(s);
h) allowing the circularization probe(s) and the denatured nucleotide-elongated fragment(s) to hybridize and form (a) circularized denatured nucleotide-elongated fragment(s);
i) optionally, removing an overhang;
j) optionally, filling in missing nucleotides between (part of) the Known Nucleotide Sequence Section and (part of) the nucleotide-elongated sequence;
k) ligating the ends of the circularized nucleotide-elongated fragment(s) to obtain (a) ligated circularized nucleotide-elongated fragment(s); and
l) sequencing the ligated circularized nucleotide-elongated fragment(s); wherein, for each fragment, sequence information of only one single Known Nucleotide Sequence section is required to obtain sequence information of the ligated circularized nucleotide-elongated fragment(s).

The three embodiments detailed hereinabove are embodiments of the same concept, but with an interchange in the steps of denaturation and combination with a circularization probe or wherein the adaptor ligation step is replaced by adding one or more, preferably 10-20, nucleotides, to the fragment as an alternative for adaptor ligation. Throughout this application many variants and embodiments of the invention are described. Some of the variants and embodiments are focussed on a specific technical feature and are only described within the realms of that feature and not directly in relation to all embodiments disclosed herein. Nevertheless, it will become clear to the skilled man, without being explicitly mentioned, that an embodiment or variant of one specific feature may and will find application analogously in other embodiment, without describing the whole method again.

The invention provides sequence data from a nucleic acid sample starting from a point where there is some sequence information already available. This may be from the same organism or it may be from another, preferably related, organism. Thus part of the sequence of the nucleic acid is known. The part of the sequence that is known can be as low as 0.01%, 0.1%, 1%, 5% or 10%. When multiple samples are investigated, the part of the sequence that is known is independent for each sample. In such an embodiment, the complete sequence of one (or more, but not from all) of the samples may be completely (i.e. 100% known). For example, when used for resequencing typically the reference sequence is known for a larger part (if not completely, i.e. 100%) in comparison to the second sequence from which only a relative small part is known or even nothing at all. Again, in the case of resequencing based on sequence information from another species, it may be that sequence information from one sample (one species, say eggplant) is (part) known and used for resequencing another species (say tomato). In such an embodiment, the origin of the KNSS is from an different species (eggplant), but is used for analysing and generating sequence information for another species (tomato). Thus, at least part of the nucleotide sequence information for the nucleic acid sample under investigation, for which more sequence information is desirable, is available in the form of at least one Known Nucleotide Sequence Section, which need not be identical. It may be that over the length of the KNSS, the percentage sequence identity is more than 50%, more than 75%, more than 90%, more than 95%, such that the circularization probe is capable of hybridising to the KNSS of the fragment under investigation.

This already available sequence information (indicated herein as Known Nucleotide Sequence Section or KNSS) can be sequence information of which also functional information is available such as gene sequences, promotors etc. But also sequence information from which no functional information is available such as partial genomes, ESTs, physical maps, fragments that have been identified in other technologies such as sequence markers, (short) sequence reads from high throughput sequencing methods such as generated by Illumina's Sequencing by Synthesis or by 454 Sequencing technologies from Roche (GSII or GS Flex) or current sequencing technologies such as generically indicated as Next-Next Generation sequencing and/or SMRT sequencing (Pacific BIO Biosciences etc. and described inter alia in Quail et al., BMC Genomics 2012, 13:341

Examples of such reads can also be AFLP derived fragments, i.e. AFLP fragments that have been at least partially sequenced.

Another examples of a source of sequence information is a WGP tag. WGP tags are sequences that have been generated using a combination of pooled BAC libraries and high through put sequencing to generate reads from which a physical map can be generated. See for instance EP534858, WO2008007951, WO2010082815A1, WO2011074960A1.

Typically, a minimum length for the Known Nucleotide Sequence Section is from 6 nucleotides. Below 6 nucleotides in length, the section becomes too short to be useful in the later development of a circularization probe due to a-specificity of annealing steps. The minimum length for the Known Nucleotide Sequence Section is preferably at least 6, at least 7, at least 8, with a preference of at least 10. Good results have been obtained with Known Nucleotide Sequence Section lengths of between 10 and 30, preferably between 12 and 25, more preferably between 15 and 20. Longer lengths are possible (up to 40, 50 or 100) and work equally well, but result in circularization probes that are relatively long and may be more cumbersome to synthesize.

The nucleic acid sample is fragmented to yield one or more fragments. The fragmentation can be achieved by physical means or by enzymatic means. Physical means comprise shearing, sonication, nebulization and the like. There is a preference for shearing. Physical means for providing fragments results in a random set of fragments of which the ends are typically not known. The length distribution of the fragments may vary with the intensity of the fragmentation process.

The enzymatic means of fragmenting the nucleic acid is by digestion with one or more nuclease enzymes, preferably a restriction endonuclease enzyme. Restriction enzymes can be used since nucleic acid samples, and hence Known Nucleotide Sequence Section may comprise restriction enzyme digestion sites, i.e. a Known Nucleotide Sequence Section may contain an restriction enzyme digestion site or a restriction enzyme digestion site may be located outside the Known Nucleotide Sequence Section.

Thus, the nucleic acid sample may contains (a) restriction enzyme digestion site(s). The presence of a restriction enzyme digestion site is maybe known from the available sequence information, but it may also be derivable from statistical analysis of the genome under investigation. Since restriction enzymes recognition sequences typically are 4-8 nucleotides long, the statistical occurrence of a recognition site will be, on average, every 256 nucleotides for a 4 bp cutter such as MseI.

The fragments of the nucleic acid sample are then provided by digesting the nucleic acid sample with the restriction endonuclease enzyme at the restriction endonuclease digestion site to yield restriction endonuclease digested fragments.

Thus, in certain embodiments, the Known Nucleotide Sequence Section comprises a restriction enzyme digestion site. A restriction enzyme typically has a recognition site, where the enzyme recognizes the relevant part of the nucleic acid, and a digestion site where the nucleic acid is cut or digested. The recognition site can be the same as the cutting site (Type II, such as EcoRI) or the cutting site can be placed further away from the recognition site (Type IIs, such as FokI).

As used herein, the term “restriction enzyme” or “restriction endonuclease” (the terms ‘restriction enzyme’ and ‘restriction endonuclease’ are used interchangeably) refers to an enzyme that recognizes a specific nucleotide sequence (recognition site) in a double-stranded DNA molecule, and will cleave both strands of the DNA molecule at or near every recognition site, leaving a blunt or a staggered end. Also encompassed are so-called nicking restriction enzymes that contain recognition sites for single or double strand DNA but subsequently cut (nick) in only one strand.

As used herein, the term “isoschizomers” refers to pairs of restriction enzymes which are specific to the same recognition sequence and which cut in the same location. For example, Sph I (GCATĜC) and Bbu I (GCATĜC) are isoschizomers of each other. The first enzyme to recognize and cut a given sequence is known as the prototype, all subsequent enzymes that recognize and cut that sequence are isoschizomers. An enzyme that recognizes the same sequence but cuts it differently is a neoschizomer. Isoschizomers are a specific type (subset) of neoschizomers. For example, Sma I (CCĈGGG) and Xma I (ĈCCGGG) are neoschizomers (not isoschizomers) of each other. Isoschizomers and neoschizomers can be used in the present invention so that the restriction enzyme that has been used in the way in which the Known Nucleotide Sequence Section was obtained need not be the same as the restriction enzyme that is used in the present method.

As used herein, the term “Class-II restriction endonuclease” refers to an endonuclease that has a recognition sequence that is located at the same location as the restriction site. In other words, Class II restriction endonucleases cleave within their recognition sequence. Examples thereof are EcoRI (G/AATTC) and Small (CCC/GGG).

As used herein, the term “Class-IIs restriction endonuclease” refers to an endonuclease that has a recognition sequence that is distant from the restriction site. In other words, Type IIs restriction endonucleases cleave outside of their recognition sequence to one side. Examples thereof are NmeAIII (GCCGAG(21/19) and FokI, AlwI.

Thus, in certain embodiments of the invention, the restriction endonuclease enzyme digestion site(s) and the restriction endonuclease enzyme recognition site(s) are located at the same position (Class II restriction endonuclease). In certain other embodiments of the invention, the restriction endonuclease enzyme digestion site(s) and the restriction endonuclease enzyme recognition site(s) are not located at the same position (Class IIS or IIB restriction endonuclease). In certain other embodiments, the restriction endonuclease enzyme digestion site(s) is located outside the restriction endonuclease enzyme recognition side on one side (Class IIS restriction endonuclease) or on both sides (Class IIB restriction endonuclease). Combinations of enzymes and combination of different classes of enzymes can be used in providing restriction fragments. Also combinations of physical fragmentation and enzymatic fragmentation can be used throughout all embodiments of the invention.

Thus the Known Nucleotide Sequence Section may comprises a restriction enzyme digestion site. The restriction enzyme digestion site (depicted herein as XXXYYY) can be located inside (internally) the Known Nucleotide Sequence Section (the other nucleotides of the Known Nucleotide Sequence Section indicated as NNNNNN) such that the entire Known Nucleotide Sequence Section can be depicted as (NNNNNNNNXXXYYYNNNNNN). It can also be located at the border of the Known Nucleotide Sequence Section (NNNNNNNNNNXXXYYY). It can be that the Known Nucleotide Sequence Section is obtained via previous methods that used restriction enzymes, such as AFLP or High throughput physical mapping such as described in WO2008007951 that provides sequence reads that can include part of the remains of a restriction enzyme digestion site (NNNNNXXX). Such fragments can also be used as Known Nucleotide Sequence Section. The structure of such a Known Nucleotide Sequence Section can be depicted as NNNNNNNNNXXXYYY, wherein N and X are as described herein elsewhere and are known from their sequence. YYY are the nucleotides that formed the other part of the restriction enzyme digestion site XXXYYY (the other half of the digestion site). Although YYY is then not directly identifiable in the AFLP fragment or the sequence read, it can nevertheless be considered as inherently present as it can be deduced from the origin of the fragment that the restriction enzyme digestion site was present in the original nucleic acid sample that generated the sequence information of which the Known Nucleotide Sequence Section. For example, if an sequenced AFLP fragment has been obtained using MseI (T/TAA) as one of the restriction enzymes and the sequence information is XXXXAAT, then the complete Known Nucleotide Sequence Section would be XXXXAATT, as T was inherently present due to the use of MseI.

The Known Nucleotide Sequence Section can be identified from the available sequence information of the nucleic acid samples by the way the information was previously obtained (for instance using restriction enzyme-based methods such as AFLP or high throughput physical mapping WO2008007951) and/or by screening the available sequence information with an algorithm that is capable of identifying restriction enzyme recognition and/or digestion sites.

The Known Nucleotide Sequence Section may be at the one of the ends of a fragment, or it may be inside the fragment and hence be removed from the ends of the fragment, the Known Nucleotide Sequence Section can located at a position removed from the ends of the fragments, preferably at a position at least 5, 10, 15, 20, 30, 50, 75 or 100 nucleotides form the ends of the fragment.

The nucleic acid sample can be digested with a restriction enzyme. The restriction enzyme digests (cuts) the nucleic acid at the restriction enzyme digestion site. The result is that restriction enzyme digested fragments are obtained. The ends of the restriction enzyme digested fragments can be blunt or staggered, depending on the restriction enzyme.

As used herein, the term “restriction enzyme digested fragment(s)” or “restriction fragment(s)” refers to the DNA molecules produced by digestion with a restriction endonuclease. Any given genome (or nucleic acid, regardless of its origin) will be digested by a particular restriction endonuclease into a discrete set of restriction fragments. The DNA fragments that result from restriction endonuclease cleavage can be further used in a variety of techniques.

The restriction fragments that can be obtained in the method of the present invention and that comprise a KNSS can have as a typical structure XXXNNNNZZZZZZYYY, wherein NNNN, XXX and YYY are as defined herein above, NNNN can be any length of the Known Nucleotide Sequence Section that is known and ZZZZZZZ is any length of the restriction fragment that is of unknown sequence and of which it is the goal to determine at least part of that sequence.

After fragmentation, whether enzymatic or physical, the fragments, in certain embodiments, can be blunted, i.e. any protruding overhangs removed. Such methods are well known in the art and the result is that the fragments have blunt ends (i.e. no overhang remains).

After fragmentation, and also after blunting, 3′ nucleotides may be added (ligated, coupled, linked) using methods known in the art (DNA polymerase) to either modify existing overhangs or to create desirable overhangs that may be used for the ligation of specific adaptors.

To at least one of the ends of the (restriction) fragments, an adaptor is ligated. Adaptors can be ligated to both ends of the (restriction) fragments and different adaptors can be provided for ligation to each end of the (restriction) fragment, for instance when Type II s enzymes are used that leave overhanging but unknown ends (like with NmeAIII (GCCGAGN(21/19) that leaves a 2 bp unknown staggered end). Different adaptors can be ligated, depending on the composition of the staggered ends.

In certain embodiments, the fragmentation, preferably by digestion with a restriction enzyme and the adaptor ligation can be performed simultaneously. When an restriction enzyme is used, the adaptor is then typically designed in such a way that the restriction site is not restored when the adaptor is ligated.

As used herein, the term “adaptors” refers to short, typically double-stranded, DNA molecules with a limited number of base pairs, e.g. about 10 to about 30 base pairs in length, which are designed such that they can be ligated to the ends of (restriction) fragments. Adaptors are generally composed of two synthetic oligonucleotides that have nucleotide sequences which are partially complementary to each other. An adaptor may have blunt ends, or may have staggered ends, or may have a blunt end and a staggered end. A staggered end is a 3′ or 5′ overhang. When mixing the two synthetic oligonucleotides in solution under appropriate conditions, they will anneal to each other forming a double-stranded structure. Adaptors can also be single stranded, in which case it may be convenient and preferred when one of the ends if the single stranded adaptor is compatible for at least a few nucleotides (2, 3, 4 or 5) with one of the strands of one of the ends of a (restriction) fragment, such that the singe stranded adaptors are capable of annealing to the (restriction) fragment. To that end a fragments may be extended by the addition of nucleotides to one of the ends of the fragment. One end of the adaptor molecule can be designed such that, after annealing, it is compatible with the end of a (restriction) fragment and can be ligated thereto; the other end of the adaptor (either in the single strand version or in the double strand version) can be designed so that it cannot be ligated, but this need not be the case, for instance when an adaptor is to be ligated in between DNA fragments, when both strands on end of the adaptor are ligatable. Being ligatable in general implies the presence of 3′-hydroxyl or 5′-phosphate groups. Being blocked from ligation generally means that the required 3′ and 5′ functionalities are lacking or blocked. In certain cases, adaptors can be ligated to fragments to provide for a starting point for subsequent manipulation of the adaptor-ligated fragment, for instance for amplification or sequencing. In the latter case, so-called sequencing adaptors may be ligated to the fragments. Being compatible for ligation can be accomplished in two (combined) ways: the end of the (double-stranded) adaptor contains an (overhanging) section that is compatible with the overhanging end of a restriction fragment such that the adaptor and the fragment may anneal. A second way is that the nucleotide that is located at the end of one strand of the adaptor is provided in such a way that it can chemically be coupled to an another nucleotide, for instance from a restriction fragment. Alternatively, a nucleotide at the end of an adaptor can also be modified (blocked) such that it cannot be coupled to another nucleotide. Double stranded adaptors may have these features combined such that the double stranded adaptor is capable of annealing to a fragment and one or both strands can be coupled to the fragment.

The adaptor (whether double or single stranded) is ligated to the end of the (restriction) fragment using a ligase. The result is an adaptor-ligated (restriction) fragment. In one embodiment, the ligation of the at least one adaptor occurs at the 5′ end of the (restriction enzyme digested) fragment(s). In one embodiment, the ligation of the at least one adaptor occurs at the 3′ end of the (restriction enzyme digested) fragment(s).

As used herein, the term “ligation” refers to the enzymatic reaction catalyzed by a ligase enzyme in which two double-stranded DNA molecules are covalently joined together. In general, both DNA strands are covalently joined together, but it is also possible to prevent the ligation of one of the two strands through chemical or enzymatic modification(s) of one of the ends of the strands. In that case the covalent joining will occur in only one of the two DNA strands.

As used herein, the term “ligating” refers to the process of joining separate (double) stranded nucleotide sequences. The double stranded DNA molecules may be blunt ended, or may have compatible overhangs (sticky overhangs) such that the overhangs can hybridize with each other. Alternatively, one of the DNA molecules may be double stranded with an overhang to which overhang another single stranded DNA molecule (single stranded adaptor) can anneal. The joining of the DNA fragments may be enzymatic, with a ligase enzyme, DNA ligase. However, a non-enzymatic, i.e. chemical ligation may also be used, as long as DNA fragments are joined, i.e. forming a covalent bond. Typically a phosphodiester bond between the hydroxyl and phosphate group of the separate strands is formed in a ligation reaction. Double stranded nucleotide sequences may have to be phosphorylated prior to ligation.

As an alternative to adaptor-ligation (whether single or double stranded), nucleotides may be added to the fragments, preferably at their 3′-end using commonly known nucleotide extension methods thereby introducing, preferably in a known order, an elongation of the fragment with a known sequence (a nucleotide elongated sequence), for instance by a sequence of steps each time introducing one nucleotide at a time (single nucleotide extension) to thereby elongate fragments with from 3-100 nucleotides, preferably from 5-50 nucleotides and with higher preference of from 18-40 nucleotides, with 10-20 nucleotides being most preferred. This elongation of fragments results in nucleotide-elongated fragments.

In embodiments of the method of the invention, the adaptor-ligated fragments are denatured. The denaturation step renders previously (party) double stranded adaptor-ligated fragments single stranded. Denaturation can be achieved by any means known the art, but typically via heating.

In the method of the present invention, a circularization probe is provided. A circularization probe is an oligonucleotide that comprises at least part of the Known Nucleotide Sequence Section and at least part of the sequence of the adaptor or at least part of the nucleotide-elongated sequence. In principle, for each fragment obtained from the fragmentation (whether by random fragmentation or restriction) of the nucleic acid sample that contains a Known Nucleotide Sequence Section, a circularization probe can be provided. For instance, when, for instance due to a sequencing protocol for the high throughput generation of a physical map (such as described in WO2008007951) 1000 sequence reads (each of these reads individually forming the basis of a Known Nucleotide Sequence Section) are obtained it is possible to generate (design) a corresponding number of circularization probes. It is also possible make a selection of these reads (a subset) for the design of circularization probes. Thus circularization probes may be provided for a selection of the Known Nucleotide Sequence Section containing denatured adaptor-ligated or nucleotide-elongated fragments. For instance, taking into account the already known distance between the reads or their distribution over the physical map, it may be convenient or preferred to select reads that are concentrated in a certain area to provide a local but thorough gap closure of the physical map. It may, alternatively or additionally, be preferred that the reads are spread out very widely over the physical map. This may also depend on the selected sequencing platform and the read length it provides. Long reads (several Kbs) may require wider spaced sequence information for the generation of Known Nucleotide Sequence Section and the circularization probes. Longer read lengths of the sequencing platform may also allow the use of restriction enzymes that generate larger fragments, i.e. have longer recognition sequences.

The part of the Known Nucleotide Sequence Section in a circularization probe can be of a length varying from 6-100 nucleotides as explained herein before. The part of the sequence of the adaptor or the nucleotide-elongated sequence in the circularization probe is at most the entire adaptor length or the nucleotide-elongated sequence length, but may be shorter such as from 8 to 30 nucleotides, preferably from 9 to 20, more preferably from 10-15 nucleotides. In the circularization probe, the Known Nucleotide Sequence Section and the adaptor sequences or the nucleotide-elongated sequences may be located adjacent. In certain embodiments, the Known Nucleotide Sequence Section and/or adaptor sequences or the nucleotide-elongated sequences may be located at (one of) the ends of the circularization probe, but there are embodiments in which there may be an overhang on one or both ends when the circularization probe is annealed to the adaptor-ligated or the nucleotide-elongated fragment.

In embodiments wherein the circularizable probe has an overhang when hybridised to the fragment, the overhang may be removed prior to ligation, preferably using an enzyme, for instance by using a flap endonuclease or a polymerase with nuclease activity, both in themselves known in the art.

The circularization probe can be directed against the bottom strand or the top strand of the denatured (single stranded) adaptor-ligated or the nucleotide-elongated fragment. Depending on whether the top or the bottom strand is targeted by the circularization probe, the orientation of the circularization probe can be different (′3-5′ vs. 5′-3′). Other adaptors, primers etc., can be modified accordingly.

In the method of the invention, the denatured (single stranded) adaptor-ligated or the nucleotide-elongated fragment is combined with the circularization probe. The combination of the single stranded adaptor-ligated or the nucleotide-elongated fragment and the circularization probe is performed under hybridizing conditions. The denatured adaptor-ligated or the nucleotide-elongated fragment and the circularization probe are allowed to hybridize. The circularization probe will anneal to the part of the Known Nucleotide Sequence Section on or near one end of the fragment and to part of the adaptor or the nucleotide—elongated on or near the other end. The hybridized single stranded adaptor-ligated or the nucleotide-elongated fragment and the circularization probe form a circular structure. The now circular structure of the single stranded adaptor-ligated or the nucleotide-elongated fragment is depicted as a circularized denatured adaptor-ligated or the nucleotide-elongated fragment. It is circularized but not yet circular as it is stabilized in its circular form by the presence of the circularization probe. It only becomes circular once the ends of the circularized probe have been ligated or otherwise connected to each other.

In an embodiment wherein the part of the Known Nucleotide Sequence Section and the part of the adaptor or nucleotide-elongated sequence are located adjacent to each other in the circularization probe, the ends of the circularized denatured adaptor-ligated or the nucleotide-elongated fragment are also located adjacent when annealed to the circularization probe. The ends of the circularized denatured adaptor-ligated or the nucleotide-elongated fragment can be ligated when located adjacent. In certain embodiments, when there is an intermittent section between the part of the Known Nucleotide Sequence Section and the part of the adaptor or the nucleotide-elongated sequence in the circularization probe such as a spacer, (an embodiment discussed more extensively elsewhere) there is a gap between the ends of the circularized denatured adaptor-ligated or nucleotide-elongated fragment that can be filled either with nucleotides or an oligonucleotide such that the (filled) circularized denatured adaptor-ligated or nucleotide-elongated fragment can be ligated to provide a ligated circularized denatured adaptor-ligated or nucleotide-elongated fragment. The ligation can be performed using a ligase or other means as described herein elsewhere for ligation.

The ligated circularized denatured adaptor-ligated or nucleotide-elongated fragment (also indicated as circular fragment) can now be sequenced to determine at least part of the sequence of the circular fragment. The sequence can be determined using any known sequence technology but with a preference for Next Generation Sequencing or current sequencing technologies such as Next-Next Generation sequencing and/or SMRT sequencing (such as technologies provided by Roche, Illumina, Helicos, Pacific Biosciences etc).

The sequence information obtained according to the method of the invention can be used, for instance through alignment, together with the sequence information already available (such as but not limited to the Known Nucleotide Sequence Section) to generate a more complete genome sequence of a sample. The sequence information obtained can also be used to generate sequence information to adjust the currently available sequence information and/or provide sequence information of a sample for which no information is available. Thus, in certain embodiments the sequence information obtained by the method of the invention is used for gap closure in genomes sequences, preferably at one or more positions where at least one Known Nucleotide Sequence Section is available. In another embodiment, the further sequence information is linked to existing sequence information such as from a physical map or a draft genome sequence. In a particular preferred embodiment the Known Nucleotide Sequence Section is linked to a region of the genome in which a (plant) trait or gene is located, for instance because the Known Nucleotide Sequence Section is obtained from a polymorphic marker such as an AFLP marker or RFLP marker or from some previous genetic marker information. It can also be used to further create an assembly of an existing physical map with the now obtained sequence information to improve the density of the physical map. As used herein, the term “assembly” refers to the construction of a contig based on ordering a collection of (partly) overlapping sequences, also called “contig building”. Further use of the method is embodied in its use in resequencing or for the determination of sequence variety in the vicinity of the Known Nucleotide Sequence Sections. Vicinity in this context is within 10000 nucleotides, preferably within 5000, 2500, 1000, 500, 250, or 100 nucleotides from the Known Nucleotide Sequence Section.

It will be clear from the context of the invention that the method can also be performed ‘in multiplex’. This means that the method works equally well with a plurality of different Known Nucleotide Sequence Sections and/or a plurality of nucleic acid samples and/or a multiplicity of restriction enzymes. Whether in monoplex format or in multiplex, the essence remains that a circularizable structure is created (where necessary after flap removal) with on one end a KNSS and an adaptor-ligated or nucleotide-elongated fragment at the other end which after ligation of the two ends is sequenced. It will also be clear that the embodiments and variations that have been described for monoplex applications as discussed herein above extensively are likewise applicable to the below multiplex options.

Hereinbelow the multiplex variants will be elaborated upon, based on the three monoplex embodiments describe hereinabove.

In one embodiment, the available part of the nucleotide sequence of the nucleic acid sample is available in the form of a plurality of Known Nucleotide Sequence Sections. Thus, in one embodiment wherein a plurality of different Known Nucleotide Sequence Sections are used, the method of the invention pertains to a method for obtaining sequence information from a nucleic acid sample, the method comprising the steps of:
a) providing a nucleic acid sample wherein at least part of the nucleotide sequence information for the nucleic acid sample is available in the form of a plurality of Known Nucleotide Sequence Sections;
b) fragmenting the nucleic acid sample to obtain one or more fragment(s);
c) optionally, blunting the ends of the fragments;
d) optionally, adding one or more 3′ nucleotides to the fragments;
e) ligating one or more adaptor(s) to one or both ends of fragment(s) to obtain adaptor-ligated fragment(s);
f) denaturing the adaptor-ligated fragment(s) to obtain denatured adaptor-ligated fragment(s);
g) providing for at least one, preferably for each, of the plurality of, optionally selected, Known Nucleotide Sequence Section, a circularization probe that comprises at least part of the Known Nucleotide Sequence Section and at least part of the sequence of the adaptor
h) combining the denatured adaptor-ligated fragment(s) with the circularization probe(s);
i) allowing the circularization probe and the denatured adaptor-ligated fragment(s) to hybridize and form circularized denatured adaptor-ligated fragment(s);
j) optionally, removing an overhang;
k) optionally, filling in missing nucleotides between (part of) the Known Nucleotide Sequence Section and (part of) the adaptor;
l) ligating the ends of the circularized adaptor-ligated fragment to obtain ligated circularized adaptor-ligated fragment(s); and
m) sequencing the ligated circularized adaptor-ligated fragment(s);
   wherein sequence information of the ligated circularized adaptor-ligated fragment(s) is obtained for each of the (selected) Known Nucleotide Sequence Sections.

The plurality of Known Nucleotide Sequence Sections and its use in the design of circularization probes provides a plurality of sequence information of ligated circularized adaptor-ligated fragment(s) for each Known Nucleotide Sequence section. In certain embodiments, the order of the steps of providing a circularizable probe, combining the adaptor-ligated probes and the denaturation step can be interchanged to the order of the denaturation step, providing a circularizable probe, and combining the adaptor-ligated probes. In certain embodiment the adaptor-ligation can be replaced by adding 3′ nucleotides to the fragment in a nucleotide elongation step. These variants are likewise applicable for the below embodiment pertaining to a multiplex variant with a plurality of samples.

In one embodiment, a plurality of samples each containing one or more Known Nucleotide Sequence Sections are analysed to thereby obtain further sequence information. Thus, in one embodiment wherein a plurality of samples are used, the method of the invention pertains to a method for obtaining sequence information from a multitude of nucleic acid samples, the method comprising the steps of:
a) providing a multitude of nucleic acid samples wherein at least part of the nucleotide sequence information of at least of the nucleic acid samples is available in the form of Known Nucleotide Sequence Section;
   for each of the nucleic acid samples, either combined or separate:
b) fragmenting the nucleic acid sample to obtain one or more fragment(s);
c) optionally, blunting the ends of the fragments;
d) optionally, adding one or more 3′ nucleotides to the fragments;
e) ligating one or more adaptor(s) to one or both ends of fragment(s) to obtain adaptor-ligated fragment(s);
f) denaturing the adaptor-ligated fragment(s) to obtain denatured adaptor-ligated fragment(s);
g) providing for at least one, preferably for each, of the plurality of, optionally selected, Known Nucleotide Sequence Section, a circularization probe that comprises at least part of the Known Nucleotide Sequence Section and at least part of the sequence of the adaptor
h) combining the denatured adaptor-ligated fragment(s) with the circularization probe(s);
i) allowing the circularization probe and the denatured adaptor-ligated fragment(s) to hybridize and form circularized denatured adaptor-ligated fragment(s);
j) optionally, removing an overhang;
k) optionally, filling in missing nucleotides between (part of) the Known Nucleotide Sequence Section and (part of) the adaptor;
l) ligating the ends of the circularized adaptor-ligated fragment to obtain ligated circularized adaptor-ligated fragment(s); and
m) sequencing the ligated circularized adaptor-ligated fragment(s);
   wherein sequence information of the ligated circularized adaptor-ligated fragment(s) is obtained for each of the (selected) Known Nucleotide Sequence Sections for each of the samples.

It is specifically observed that in certain embodiments, the multiplex methods as described herein above using multiple KNSS and/or multiple samples and/or multiple restriction enzymes are also provided based on the use of a 3′-nucleotide-elongated fragment or with the denaturation step and the step of combining with the circularization probe interchanged.

In one of its most simple forms based on the use of restriction enzymes, the invention pertains to a method for obtaining sequence information from a nucleic acid sample, the method comprising the steps of:
a) providing a nucleic acid sample wherein at least part of the nucleotide sequence information for the nucleic acid sample is available in the form of a Known Nucleotide Sequence Section, wherein each Known Nucleotide Sequence Section comprises one or more restriction enzyme digestion site(s);
b) digesting the nucleic acid sample with a restriction enzyme wherein the restriction enzyme digests at the restriction enzyme digestion site to obtain restriction-enzyme digested fragment(s);
c) ligating an adaptor to one or both of the restriction-enzyme digested ends of the restriction-enzyme digested fragment(s) to obtain adaptor-ligated restriction-enzyme digested fragment(s);
d) denaturing the adaptor-ligated restriction-enzyme digested fragment(s) to obtain denatured adaptor-ligated restriction-enzyme digested fragment(s);
e) providing, preferably for each fragment, a circularization probe that comprises at least part of the Known Nucleotide Sequence Section and at least part of the sequence of the adaptor
f) combining the denatured adaptor-ligated restriction-enzyme digested fragment(s) with the circularization probe
g) allowing the circularization probe and the denatured adaptor-ligated restriction-enzyme digested fragment(s) to hybridize and form circularized denatured adaptor-ligated restriction-enzyme digested fragment(s);
h) ligating the ends of the circularized adaptor-ligated restriction-enzyme digested fragment to obtain ligated circularized adaptor-ligated restriction-enzyme digested fragment(s); and
i) sequencing the ligated circularized adaptor-ligated restriction-enzyme digested fragment(s);
   wherein, for each fragment, sequence information of only one single Known Nucleotide Sequence section is required to obtain sequence information of the ligated circularized adaptor-ligated restriction-enzyme digested fragment(s).

In one embodiment, the available part of the nucleotide sequence of the nucleic acid sample is available in the form of a plurality of Known Nucleotide Sequence Sections that comprise a restriction enzyme digestion site. Thus, in one embodiment wherein a plurality of different Known Nucleotide Sequence Sections are used, the method of the invention pertains to a method for obtaining sequence information from a nucleic acid sample, the method comprising the steps of:
a) providing a nucleic acid sample wherein at least part of the nucleotide sequence information for the nucleic acid sample is available in the form of a plurality of Known Nucleotide Sequence Sections, wherein each Known Nucleotide Sequence Section comprises a restriction enzyme digestion site;
b) digesting the nucleic acid sample with one or more restriction enzyme(s) wherein the restriction enzyme(s) digest(s) at the restriction enzyme digestion site(s) to obtain restriction-enzyme digested fragment(s);
c) ligating one or more adaptor(s) to one or both of the restriction-enzyme digested ends of the restriction-enzyme digested fragment(s) to obtain adaptor-ligated restriction-enzyme digested fragment(s);
d) denaturing the adaptor-ligated restriction-enzyme digested fragment(s) to obtain denatured adaptor-ligated restriction-enzyme digested fragment(s);
e) providing a circularization probe that comprises at least part of the Known Nucleotide Sequence Section and at least part of the sequence of the adaptor
f) combining the denatured adaptor-ligated restriction-enzyme digested fragment(s) with the circularization probe
g) allowing the circularization probe and the denatured adaptor-ligated restriction-enzyme digested fragment(s) to hybridize and form circularized denatured adaptor-ligated restriction-enzyme digested fragment(s);
h) ligating the ends of the circularized adaptor-ligated restriction-enzyme digested fragment to obtain ligated circularized adaptor-ligated restriction-enzyme digested fragment(s); and
i) sequencing the ligated circularized adaptor-ligated restriction-enzyme digested fragment(s);
   wherein sequence information of only one single Known Nucleotide Sequence Section is required to obtain sequence information of the ligated circularized adaptor-ligated restriction-enzyme digested fragment(s) for each of the Known Nucleotide Sequence Sections.

In one embodiment, a plurality of samples each containing one or more Known Nucleotide Sequence Sections are analysed to thereby obtain further sequence information. Thus, in one embodiment wherein a plurality of samples are used, the method of the invention pertains to a method for obtaining sequence information from a multitude of nucleic acid samples, the method comprising the steps of:
a) providing a multitude of nucleic acid samples wherein at least part of the nucleotide sequence information of the nucleic acid samples is available in the form of Known Nucleotide Sequence Section, wherein each Known Nucleotide Sequence Section comprises a restriction enzyme digestion site;
   for each of the nucleic acid samples, either combined or separate:
b) digesting the nucleic acid sample with a restriction enzyme wherein the restriction enzyme digests at the restriction enzyme digestion site to obtain restriction-enzyme digested fragment(s);
c) ligating an adaptor to at least one of the restriction-enzyme digested ends of the restriction-enzyme digested fragment(s) to obtain adaptor-ligated restriction-enzyme digested fragment(s);
d) denaturing the adaptor-ligated restriction-enzyme digested fragment(s) to obtain denatured adaptor-ligated restriction-enzyme digested fragment(s);
e) providing circularization probes for each of the plurality of Known Nucleotide Sequence Sections, wherein each circularization probe comprises at least part of one a Known Nucleotide Sequence Section and at least part of the sequence of the adaptor;
f) combining the denatured adaptor-ligated restriction-enzyme digested fragment(s) with the circularization probes allowing the circularization probe and the denatured adaptor-ligated restriction-enzyme digested fragment(s) to hybridize and form circularized denatured adaptor-ligated restriction-enzyme digested fragment(s);
g) ligating the ends of the circularized adaptor-ligated restriction-enzyme digested fragment to obtain ligated circularized adaptor-ligated restriction-enzyme digested fragment(s); and
h) sequencing the ligated circularized adaptor-ligated restriction-enzyme digested fragment(s);

The Known Nucleotide Sequence Section(s) may be the same for each sample (thereby allowing polymorphism screening between samples by comparing the obtained sequence information) or may be different (for instance to generate as much sequence information as possible).

The samples may be combined into a pool of samples, basically at any point in the method, already from the beginning or may be processed separately up and including the sequencing step. They may be combined after the adaptor ligation step, or after the circularization step.

If samples are processed together, for instance when pooled or otherwise combined, the samples may be distinguished from each other by the incorporation of an identifier. Such an identifier can be incorporated in the adaptor and can be included already in the adaptor-ligation step, either by incorporation in the adaptor or by a separate ligation step prior or after adaptor ligation. The identifier may also be incorporated in the design of the circularization probe and can be located between the part of the Known Nucleotide Sequence Section and the part of the adaptor. The identifier can also be built in during the adding of 3′ nucleotides to obtain nucleotide-elongated fragments.

In one embodiment wherein a multiplicity of restriction enzymes are used, the method of the invention pertains to a method for obtaining sequence information from a nucleic acid sample, the method comprising the steps of:
a) providing a nucleic acid sample wherein at least part of the nucleotide sequence information of the nucleic acid sample is available in the form of Known Nucleotide Sequence Section, wherein each Known Nucleotide Sequence Section comprises one or more restriction enzyme digestion site(s);
b) digesting the nucleic acid sample with the multitude of restriction enzymes wherein the restriction enzymes digest at the respective restriction enzyme digestion sites to obtain restriction-enzyme digested fragment(s);
c) ligating an adaptor to at least one of the restriction-enzyme digested ends of the restriction-enzyme digested fragment(s) to obtain adaptor-ligated restriction-enzyme digested fragment(s);
d) denaturing the adaptor-ligated restriction-enzyme digested fragment(s) to obtain denatured adaptor-ligated restriction-enzyme digested fragment(s);
e) providing circularization probes for each of the plurality of Known Nucleotide Sequence Sections, wherein each circularization probe comprises at least part of one a Known Nucleotide Sequence Section and at least part of the sequence of the adaptor;
f) combining the denatured adaptor-ligated restriction-enzyme digested fragment(s) with the circularization probes allowing the circularization probe and the denatured adaptor-ligated restriction-enzyme digested fragment(s) to hybridize and form circularized denatured adaptor-ligated restriction-enzyme digested fragment(s);
g) ligating the ends of the circularized adaptor-ligated restriction-enzyme digested fragment to obtain ligated circularized adaptor-ligated restriction-enzyme digested fragment(s); and
h) sequencing the ligated circularized adaptor-ligated restriction-enzyme digested fragment(s).

When using a multiplicity of restriction enzymes (preferably at least two, two, at least three or three restriction enzymes), a different set of fragments that may have a different length distribution can be obtained. To fragments originating from different restriction enzymes that contain different recognition sequences, different adaptors can be ligated. So to one fragment obtained by two restriction enzymes (say EcoRI and MseI), two different adaptors can be ligated (say an EcoRI adaptor and a MseI adaptor). This can also be useful to accommodate different sequencing platforms. It is also very advantageously in improving high throughput capacity. By using different (single or double stranded) adaptors, different circularization probes can be designed. In an embodiment using different adaptors for one fragment, the circularization probe can be designed for one adaptor and the Known Nucleotide Sequence Section for one strand (for example the Top strand) and for the other adaptor and the same Known Nucleotide Sequence Section for the other strand (here the Bottom strand), thereby further increasing efficiency and reliability (determining both top and bottom strand in one sample reduces the error rate considerably).

Having different circularization probes available also allows for the selection of fragments from among a larger group and as such a complexity reduction can be achieved that may help in accommodating large samples or to aid in using the method when there is a large number of Known Nucleotide Sequence Sections (for instance when there are a large (thousands) number of sequence reads available from a physical map (see for instance WO200500791 where the present inventors generated a physical map based on several million sequence reads of about 60 nucleotides each. Parts of each of these reads may form the basis of a Known Nucleotide Sequence Section.

It will be clear from the above variations that there are combinations possible such as a multiplicity of enzymes used in combinations with a plurality of Known Nucleotide Sequence Sections. Or a plurality of Known Nucleotide Sequence Sections and a multitude of samples, etc. In this respect it is also observed that the term ‘multiplicity’, ‘multitude’, ‘plurality’ have the same meaning in that they refer to ‘more than one’ or ‘one or more’ or ‘at least one’. The different terms ‘multiplicity’, ‘multitude’, ‘plurality’ are used to create a clear picture of the various (and complex) multiplicity levels of the present invention. The different terms are intended to avoid confusion. This also means that they can be used interchangeably. This may require linguistic adaptations of the wording, but nevertheless remains within the scope of the present invention. In this respect, as used herein, the terms “a”, “an”, and “the”, in their singular forms, refer to plural referents and vice versa unless the context clearly dictates otherwise. For example, a method for isolating “a” DNA molecule, includes isolating a plurality of molecules (e.g. 10's, 100's, 1000's, 10's of thousands, 100's of thousands, millions, or more molecules).

As used herein, the terms “high throughput sequencing” and “next generation sequencing” refer to sequencing technologies that are capable of generating a large amount of reads, typically in the order of many thousands (i.e. ten or hundreds of thousands) or millions of sequence reads rather than a few hundred at a time. High throughput sequencing is distinguished over and distinct from conventional Sanger or capillary sequencing. Typically, the sequenced products are the sequenced products themselves which typically have relative short reads, between about 600 and 30 bp. Examples of such methods are given by the pyrosequencing-based methods disclosed in WO 03/004690, WO 03/054142, WO 2004/069849, WO 2004/070005, WO 2004/070007, and WO 2005/003375, by Seo et al. (2004) Proc. Natl. Acad. Sci. USA 101:5488-93. These technologies further comprise extensive and elaborate data storage and processing workflows for read assembly etc. The availability of high throughput sequencing requires many conventional workflows and methods for the analysis of genomes to be redesigned to accommodate the type and quality of data that are now produced. Next generation high throughput sequencing is extensively described also in “Next Generation Genome sequencing” M. Janitz Ed. (Wiley-Blackwell, 2008).

The circularization probe may further comprise a spacer. A spacer is a nucleotide sequence that is incorporated in the circularization probe. The spacer may be incorporated between the part of the Known Nucleotide Sequence Section and the part of the sequence of the adaptor or nucleotide-elongated sequence. The spacer can be single stranded or double stranded. The spacer can be any length. The spacer may contain also other functionalities such as a primer sequence (In general, a primer sequence is capable of binding a primer as a start for amplification or elongation) such as amplification primer sequence and/or sequencing primer sequence. The spacer may contain functionalities that are provided in separate sections of the spacer or may combine such functionalities in one (i.e. a combined amplification primer sequence that at another point in the process can be used as a sequencing primer).

A gap between the ends of the circularized fragment can be filled by a combination of polymerase with nucleotides or by an oligonucleotide or a combination thereof.

The spacer sequence or the adaptor or the nucleotide-elongated sequence or a primer may contain an identifier. An identifier can be sample-specific, Known Nucleotide Sequence Section-specific or a combination of both.

As used herein, the term “identifier” refers to a short sequence that can be added to an adaptor or a primer or included in its sequence or otherwise used as label to provide a unique identifier. Such a sequence identifier (tag) can be a unique base sequence of varying but defined length, typically from 4-16 bp used for identifying a specific nucleic acid sample. For instance 4 bp tags allow 4(exp4)=256 different tags. Using such an identifier, the origin of a sequence or sample can be determined upon further processing. In the case of combining processed products originating from different nucleic acid samples, the different nucleic acid samples are generally identified using different identifiers. Identifiers preferably differ from each other by at least two base pairs and preferably do not contain two identical consecutive bases to prevent misreads. Identifiers that differ from each other by at least two base pairs and/or do not contain two identical consecutive bases typically are longer (up from 5, so 5, 6, 7 8 or longer such as 9 or 10 nucleotides) in order to provide an adequate number of identifiers for unique identification. The identifier function can in embodiments be combined with other functionalities such as adaptors or primers, i.e. identifier-containing adaptors or primers that contain an identifier for instance 5′ of the annealing end to introduce identifiers during an amplification round.

As used herein, the term “hybridization” refers to a process which involves the annealing of a complementary sequence to the target nucleic acid. The ability of two polymers of nucleic acid containing complementary sequences to find each other and anneal through base pairing interaction is a well-recognized phenomenon. The initial observations of the “hybridization” process by Marmur and Lane, Proc. Natl. Acad. Sci. USA 46:453 (1960) and Doty et al., Proc. Natl. Acad. Sci. USA 46:461 (1960) have been followed by the refinement of this process into an essential tool of modem biology. An example of two complementary sequences is: 5′-AGTCC-3′ and 3′-GGACT-5″, wherein an A can base pair, i.e. forms hydrogen bonds, with a T, and a G with a C, in this example the two complementary from base pairs between all nucleotides, but this does not necessarily need to be the case. As long as two complementary sequences can form basepairs and anneal, the two complementary sequences are hybridized.

As used herein, the term “stringent hybridisation conditions” refers to a process used to identify nucleotide sequences, which are substantially identical to a given nucleotide sequence. The stringency of the hybridization conditions are sequence dependent and will be different in different circumstances. Generally, stringent conditions are selected to be about 5° C. lower than the thermal melting point (Tm) for the specific sequences at a defined ionic strength and pH. The Tm is the temperature (under defined ionic strength and pH) at which 50% of the target sequence hybridises to a perfectly matched probe. Typically stringent conditions will be chosen in which the salt (NaCl) concentration is about 0.02 molar at pH 7 and the temperature is at least 60° C. Lowering the salt concentration and/or increasing the temperature increases stringency. Stringent conditions for RNA-DNA hybridisations (Northern blots using a probe of e.g. 100 nt) are for example those which include at least one wash in 0.2×SSC at 63° C. for 20 min, or equivalent conditions. Stringent conditions for DNA-DNA hybridisation (Southern blots using a probe of e.g. 100 nt) are for example those which include at least one wash (usually 2) in 0.2×SSC at a temperature of at least 50° C., usually about 55° C., for 20 min, or equivalent conditions. See also Sambrook et al. (1989) and Sambrook and Russell (2001).

Hybridizing conditions as used herein are preferably high stringency conditions “High stringency” conditions can be provided, for example, by hybridization at 65° C. in an aqueous solution containing 6×SSC (20×SSC contains 3.0 M NaCl, 0.3 M Na-citrate, pH 7.0), 5×Denhardt's (100×Denhardt's contains 2% Ficoll, 2% Polyvinyl pyrollidone, 2% Bovine Serum Albumin), 0.5% sodium dodecyl sulphate (SDS), and 20 μg/ml denaturated carrier DNA (single-stranded fish sperm DNA, with an average length of 120-3000 nucleotides) as non-specific competitor. Following hybridization, high stringency washing may be done in several steps, with a final wash (about 30 min) at the hybridization temperature in 0.2-0.1×SSC, 0.1% SDS.

“Moderate stringency” refers to conditions equivalent to hybridization in the above described solution but at about 60-62° C. In that case the final wash is performed at the hybridization temperature in 1×SSC, 0.1% SDS.

“Low stringency” refers to conditions equivalent to hybridization in the above described solution at about 50-52° C. In that case, the final wash is performed at the hybridization temperature in 2×SSC, 0.1% SDS. See also Sambrook et al. (1989) and Sambrook and Russell (2001).

The adaptor-ligated fragments as well as the nucleotide-elongated fragments may be amplified. Amplification can be performed on adaptor-ligated or nucleotide-elongated fragments prior to or as part of the sequencing process. Thus the adaptor-ligated or nucleotide-elongated fragments may be amplified and/or the circularized fragments may be amplified.

Amplification may be performed using a random primer, i.e. a primer or set of primers that contain random sequences to initiate amplification. The primer for amplification may be a primer that is capable of annealing to (and initiating amplification from) at least part of the sequence of the Known Nucleotide Sequence Section or to at least part of the adaptor/nucleotide-elongated sequence, or to both. The random primer may also be designed such that it anneals to the internal sequence of the fragment, i.e. the unknown part.

Amplification may be performed using a single primer, a pair of primers or a plurality of primers. The primers may also be specific, i.e. designed to specifically amplify certain (selected) sequences, such as certain KNSS's form amongst a larger group of KNSS's.

The amplification may also be a selective amplification method such as AFLP type selective amplification. As used herein, the term “AFLP” refers to a method for selective amplification of nucleic acids based on digesting a nucleic acid with one or more restriction endonucleases to yield restriction fragments, ligating adaptors to the restriction fragments and amplifying the adaptor-ligated restriction fragments with at least one primer that is (partly) complementary to the adaptor, (partly) complementary to the remains of the restriction endonuclease, and that further contains at least one randomly selected nucleotide from amongst A, C, T, or G (or U as the case may be) at the 3′-end of the primer. AFLP does not require any prior sequence information and can be performed on any starting DNA. In general, AFLP comprises the steps of:
(a) digesting a nucleic acid, in particular a DNA or cDNA, with one or more specific restriction endonucleases, to fragment the DNA into a corresponding series of restriction fragments;
(b) ligating the restriction fragments thus obtained with a (single or double-stranded) synthetic oligonucleotide adaptor, one end of which is compatible with one or both of the ends of the restriction fragments, to thereby produce adaptor-ligated, restriction fragments of the starting DNA;
(c) contacting the adaptor-ligated, restriction fragments under hybridizing conditions with one or more oligonucleotide primers that contain selective nucleotides at their 3′-end;
(d) amplifying the adaptor-ligated, restriction fragment hybridized with the primers by PCR or a similar technique so as to cause further elongation of the hybridized primers along the restriction fragments of the starting DNA to which the primers hybridized; and
(e) detecting, identifying or recovering the amplified or elongated DNA fragment thus obtained.

AFLP type amplification thus provides a reproducible subset of adaptor-ligated fragments. AFLP is described in EP534858, U.S. Pat. No. 6,045,994 and in Vos et al 1995. AFLP: a new technique for DNA fingerprinting. Nucleic Acids Research 23(21): 4407-4414. Reference is made to these publications for further details regarding AFLP. The AFLP is commonly used as a complexity reduction technique and a DNA fingerprinting technology.

As used herein, the terms “selective base”, “selective nucleotide”, and “randomly selective nucleotide” refer to a base or a nucleotide located at the 3′ end of the primer, the selective base is randomly selected from amongst A, C, T or G (or U as the case may be). By extending a primer with a selective base, the subsequent amplification will yield only a reproducible subset of the adaptor-ligated restriction fragments, i.e. only the fragments that can be amplified using the primer carrying the selective base. Selective nucleotides can be added to the 3′ end of the primer in a number varying between 1 and 10. Typically, 1-4 suffice. Both primers (in PCR) may contain a varying number of selective bases. With each added selective base, the subset reduces the amount of amplified adaptor-ligated restriction fragments in the subset by a factor of about 4. this type of complexity reduction is considered random as it does not require or take into account any previous sequence knowledge, it is only based on the selective nucleotide. Typically, the number of selective bases used in the AFLP technology (EP534858) is indicated by +N+M, wherein one primer carries N selective nucleotides and the other primers carries M selective nucleotides. Thus, an Eco/Mse+1/+2 AFLP is shorthand for the digestion of the starting DNA with EcoRI and MseI, ligation of appropriate adaptors and amplification with one primer directed to the EcoRI restricted position carrying one selective base and the other primer directed to the MseI restricted site carrying 2 selective nucleotides. A primer used in AFLP that carries at least one selective nucleotide at its 3′ end is also depicted as an AFLP-primer. Primers that do not carry a selective nucleotide at their 3′ end and which in fact are complementary to the adaptor and the remains of the restriction site are sometimes indicated as AFLP+0 primers. The term selective nucleotide is also used for nucleotides of the target sequence that are located adjacent to the adaptor section and that have been identified by the use of selective primer as a consequence of which, the nucleotide has become known.

For the amplification of the ligated circularized fragments of the present invention, it is preferred that a polymerase is used with strand displacement activity, such as phi29. It is further preferred that the amplification is rolling circle amplification.

The amplification, whether a (selective) amplification of adaptor-ligated or nucleotide-elongated fragments (which may be linear or exponential) for enrichment or the amplification of the circularised fragment yields amplicons.

As used herein, the terms “amplification” and “amplifying” refer to a polynucleotide amplification reaction, namely, a population of polynucleotides that are replicated from one or more starting sequences. Amplifying may refer to a variety of amplification reactions, including, but not limited to, polymerase chain reaction, linear polymerase reactions, nucleic acid sequence-based amplification, rolling circle amplification and like reactions. Typically, amplification primers are used for amplification, the result of the amplification reaction being an amplicon. As used herein, the term “amplification primers” refers to single stranded nucleotide sequences which can prime the synthesis of DNA. DNA polymerase cannot synthesize DNA de novo without primers. An amplification primer hybridises to the DNA, i.e. base pairs are formed. Nucleotides that can form base pairs, that are complementary to one another, are e.g. cytosine and guanine, thymine and adenine, adenine and uracil, guanine and uracil. The complementarity between the amplification primer and the existing DNA strand does not have to be 100%, i.e. not all bases of a primer need to base pair with the existing DNA strand. The sequence of the existing DNA strand, e.g. sample DNA or an adaptor-ligated DNA fragment, to which an amplification primer (partially) hybridises is often referred to as primer binding site or primer binding sequence (PBS). From the 3′-end of a primer hybridised with the existing DNA strand, nucleotides are incorporated using the existing strand as a template (template-directed DNA synthesis). We may also refer to the synthetic oligonucleotide molecules which are used in an amplification reaction as “primers”. The newly synthesized nucleotide sequences in the amplification reaction may be referred to as being internal sequences. In case a PCR reaction is performed, the internal sequence typically is the sequence in between the two primer binding sites. According to the invention, a primer can be used in an amplification step to introduce additional sequences to the DNA. This can be achieved by providing primers with additional sequences such as an identifier, a sequencing adaptor or a capturing ligand such as a biotin moiety. Modifications can be introduced by providing them at the 5′-end of the primer, upstream from the part of the primer that enables to prime the synthesis of DNA.

As used herein, the term “amplicon” refers to the product of a polynucleotide amplification reaction, namely, a population of polynucleotides that are replicated from one or more starting sequences. Amplicons may be produced by a variety of amplification reactions, including, but not limited to, polymerase chain reactions, linear polymerase reactions, nucleic acid sequence-based amplification, rolling circle amplification and the like reactions.

In one embodiment of the invention, the ligated, circularized adaptor-ligated or nucleotide-elongated fragments or the ligated, circularized adaptor-ligated restriction enzyme digested fragments (circularized fragments) are further fragmented prior to the sequencing step. This can be advantageous if the circularized fragments are very large and exceed the read length that can be provided by the available sequencing technology. The further fragmentation can be achieved by restriction with another restriction enzyme or by physical methods such as shearing and/or nebulization, and/or nuclease treatment.

In certain embodiments, an exonuclease treatment can be performed, preferably after the circularization. The exonuclease treatment can be used to remove non-circularized sequences, i.e. sequences that have remained linear.

In certain embodiments, the circularization probe is provided with a capturing unit (biotin). Alternatively, the amplification primer can be biotinylated to capture the circularized fragment or the amplicons thereof prior to sequencing.

### BRIEF DESCRIPTION OF DRAWINGS

FIG. 1: Schematic representation of Single sample—Single KNSS—Single Restriction enzyme—Single adaptor.

Single Known Nucleotide Sequence Section sequence detection using an adaptor that ligates to the top strand of the restriction fragment. DNA is digested using a restriction enzyme (EcoRI). An adaptor is ligated and the ligation products are denatured. The denatured products are circularized using an oligonucleotide that is homologous to the adaptor sequence and the Known Nucleotide Sequence Section sequence. The ends of the circularized and denatured products are ligated. The generated ligated products are sequenced with which the Known Nucleotide Sequence Section sequence and flanking sequence information is determined.

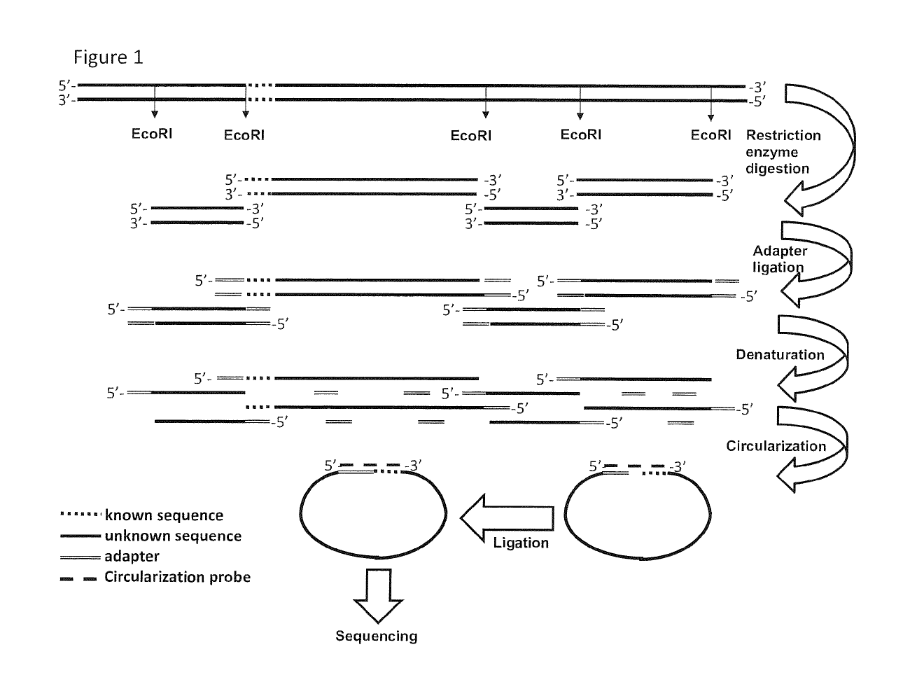

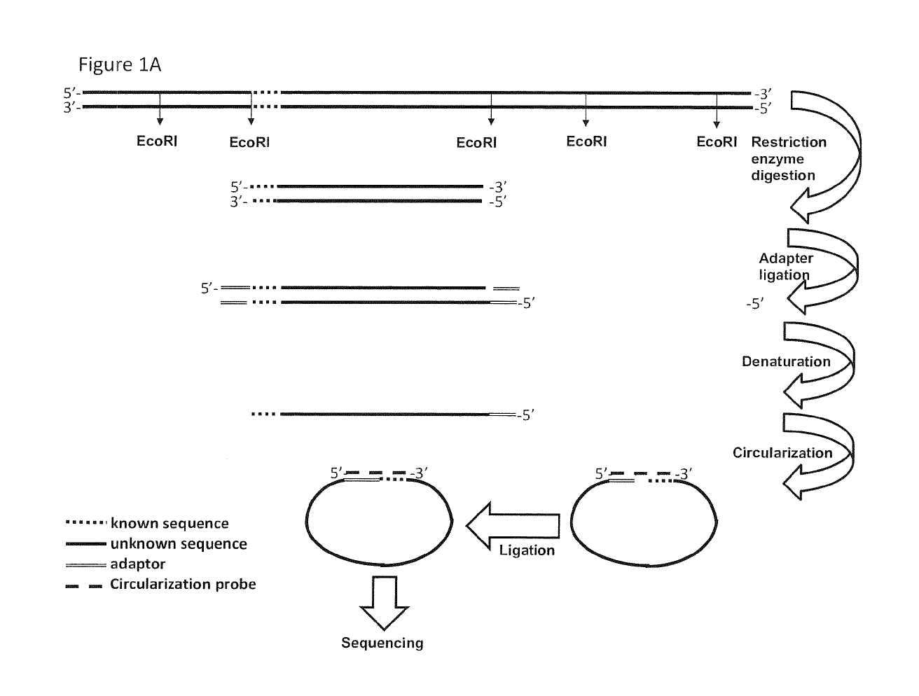

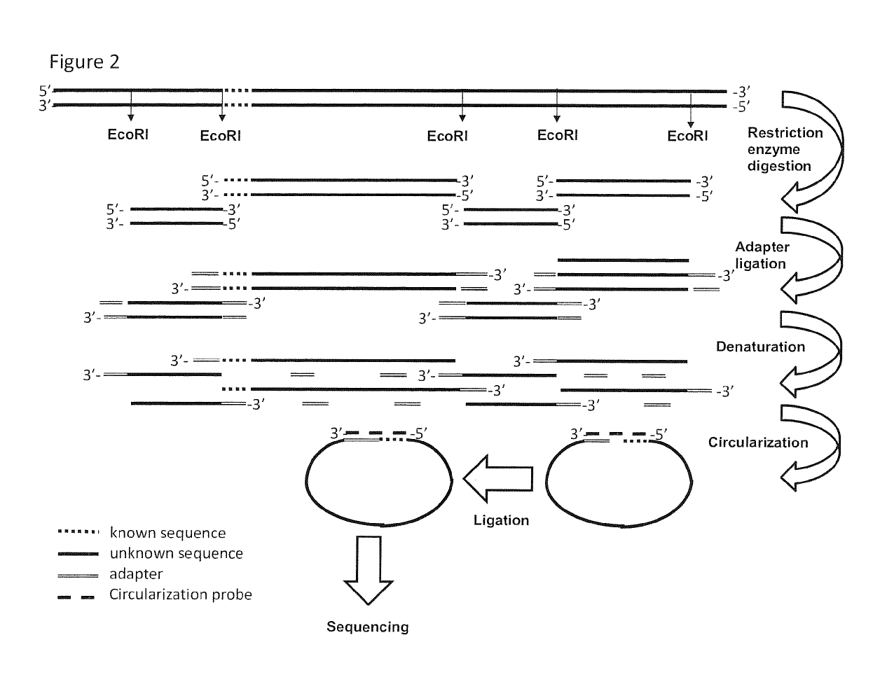

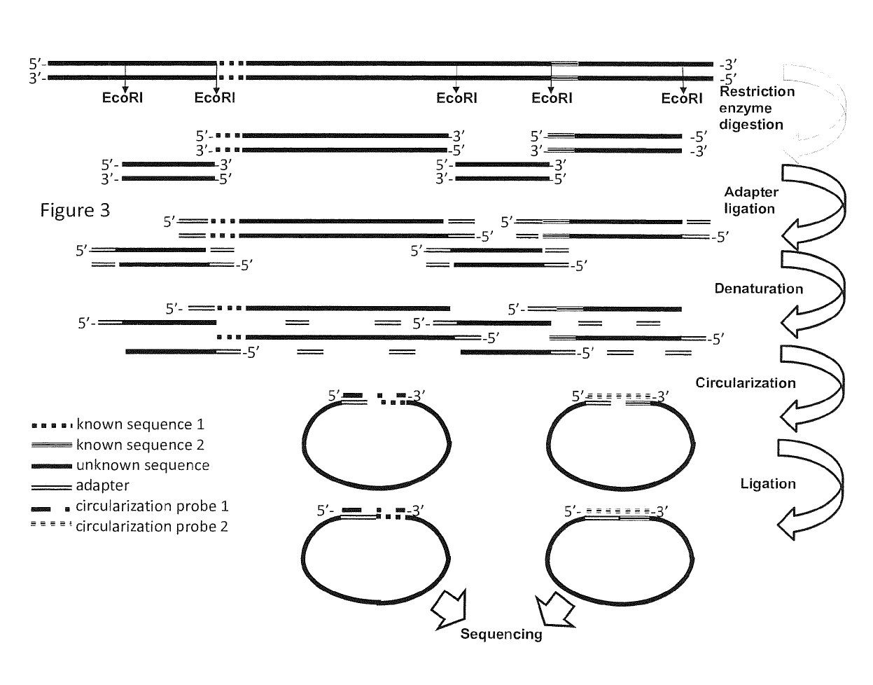

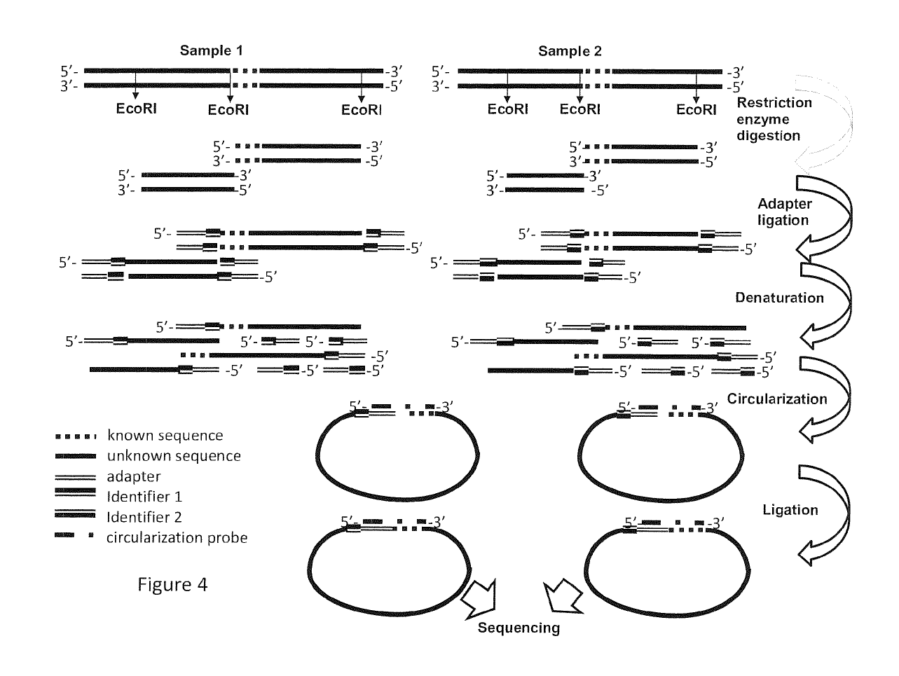

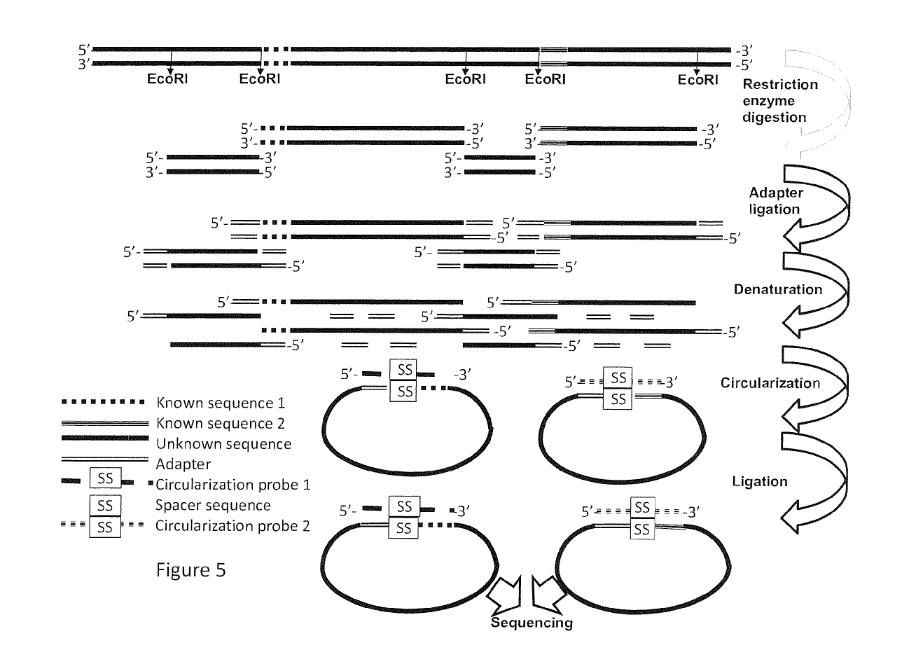

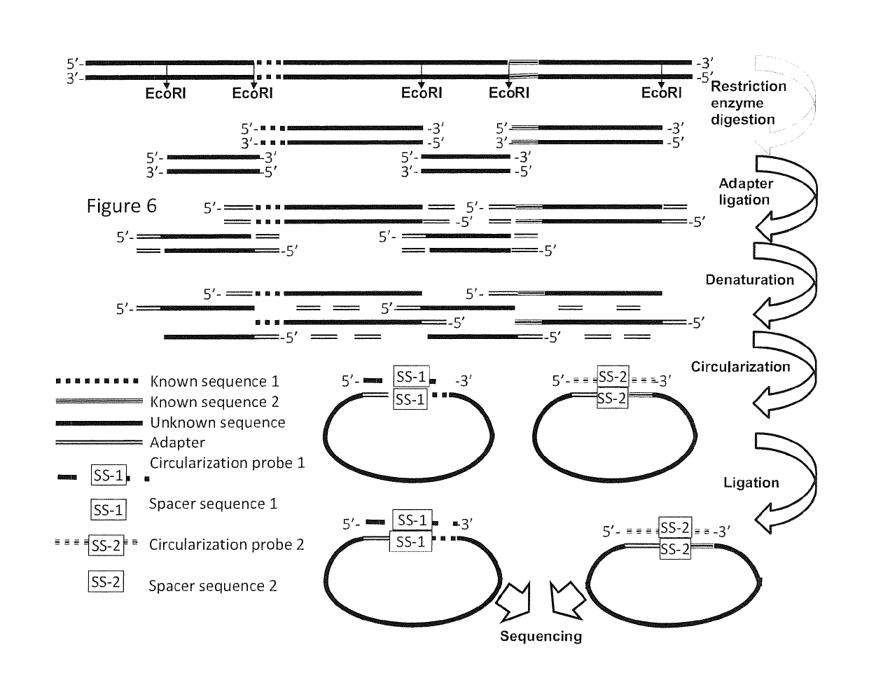

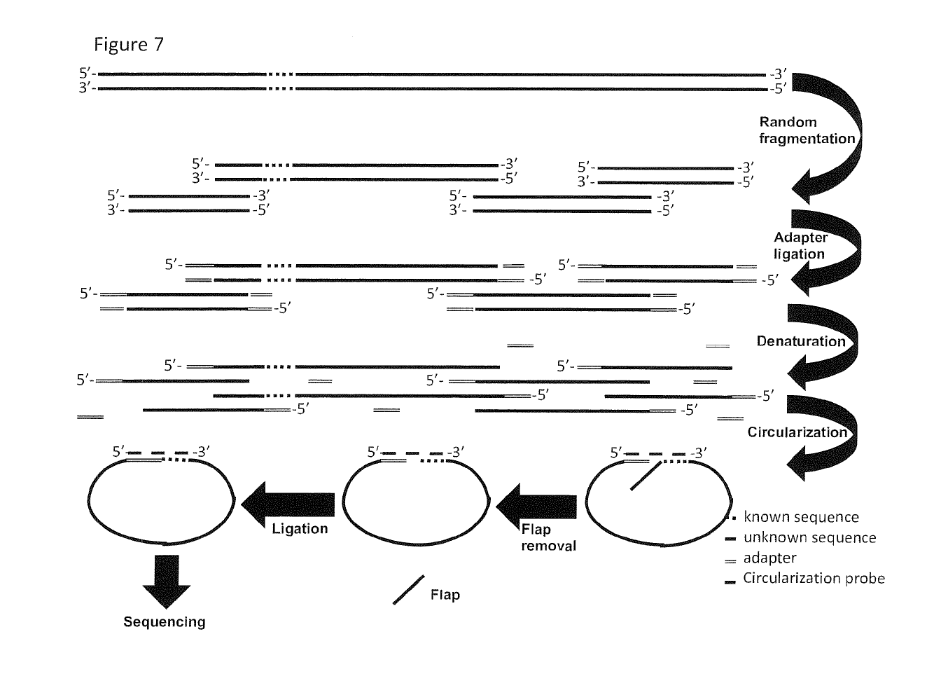

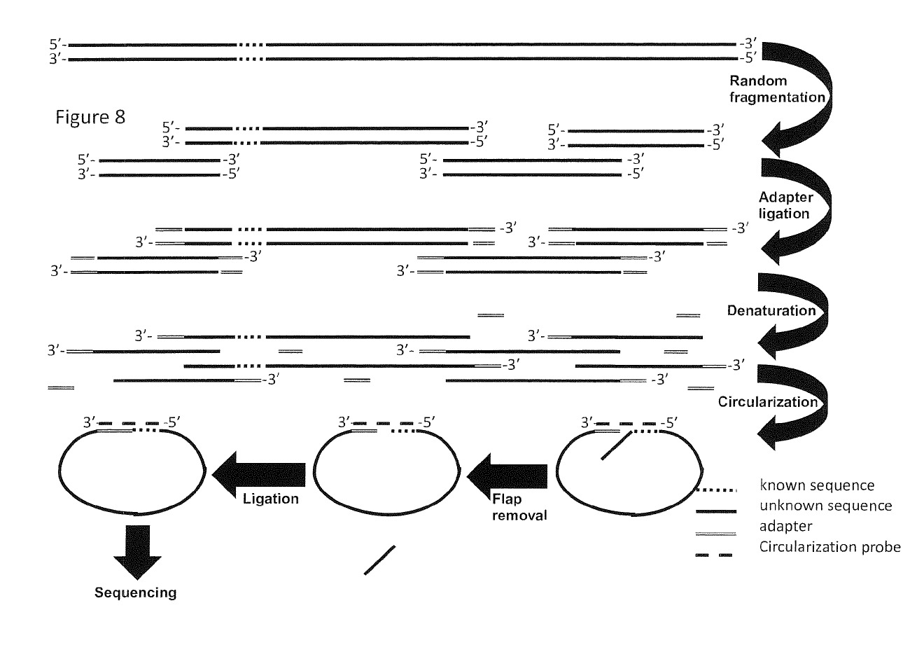

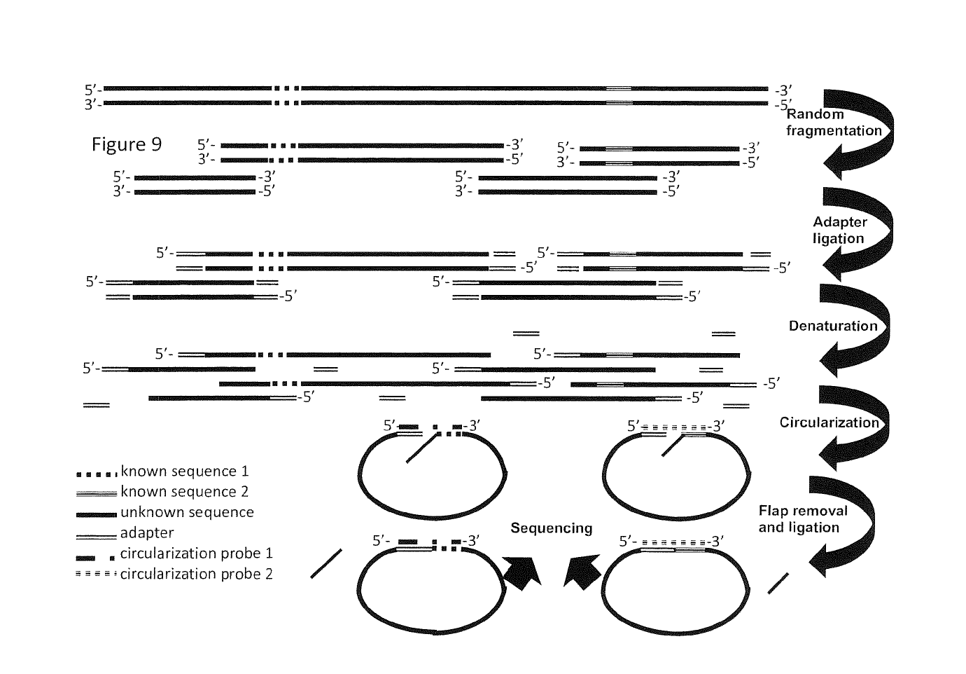

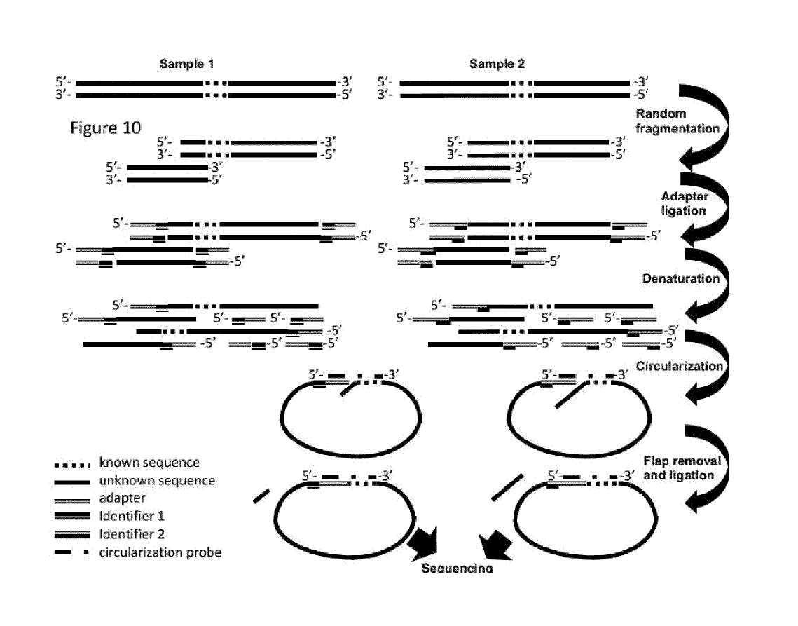

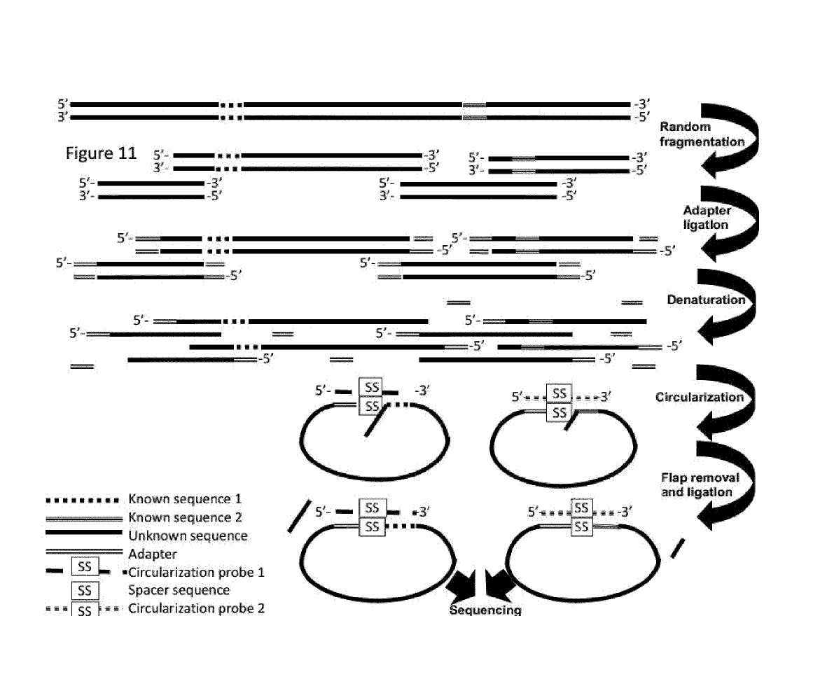

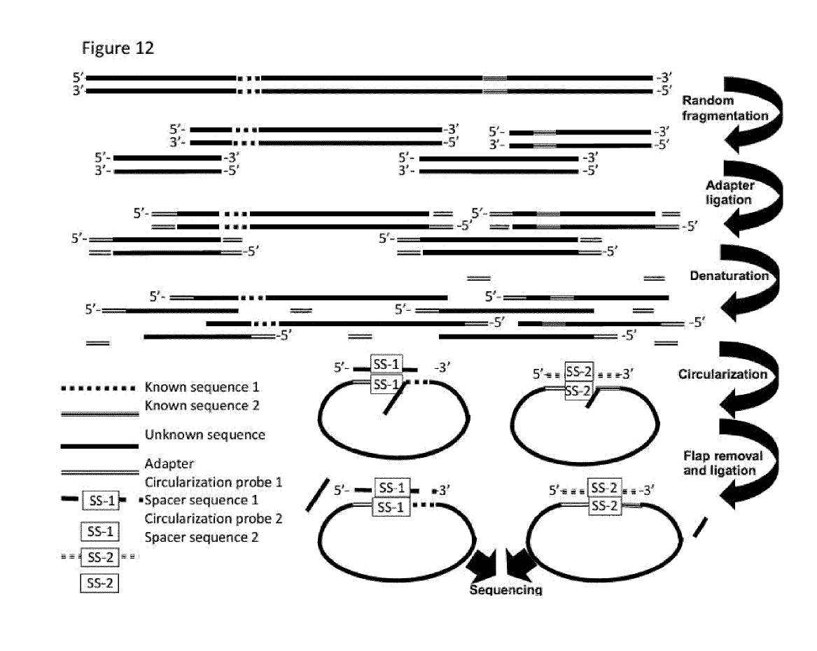

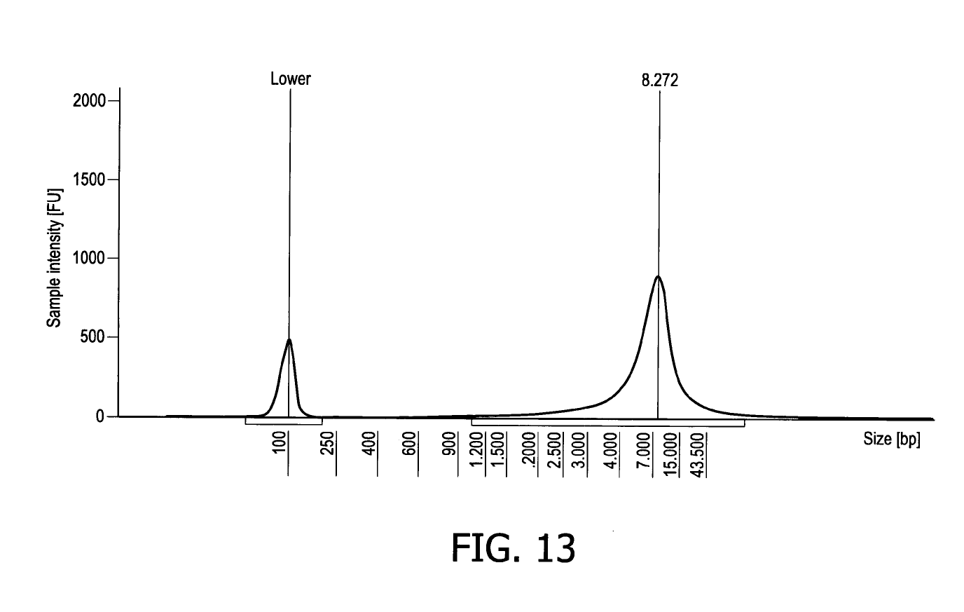

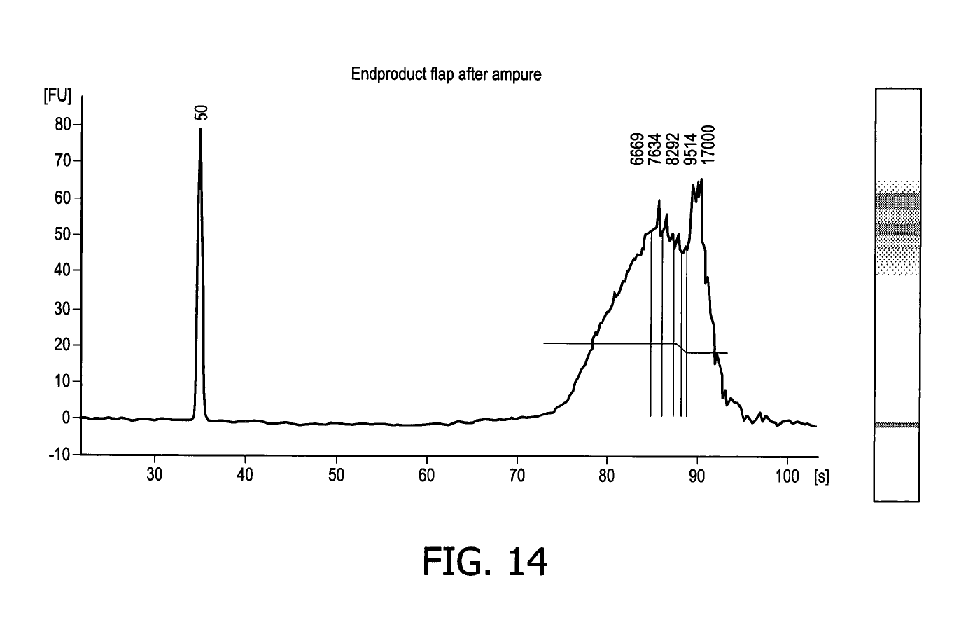

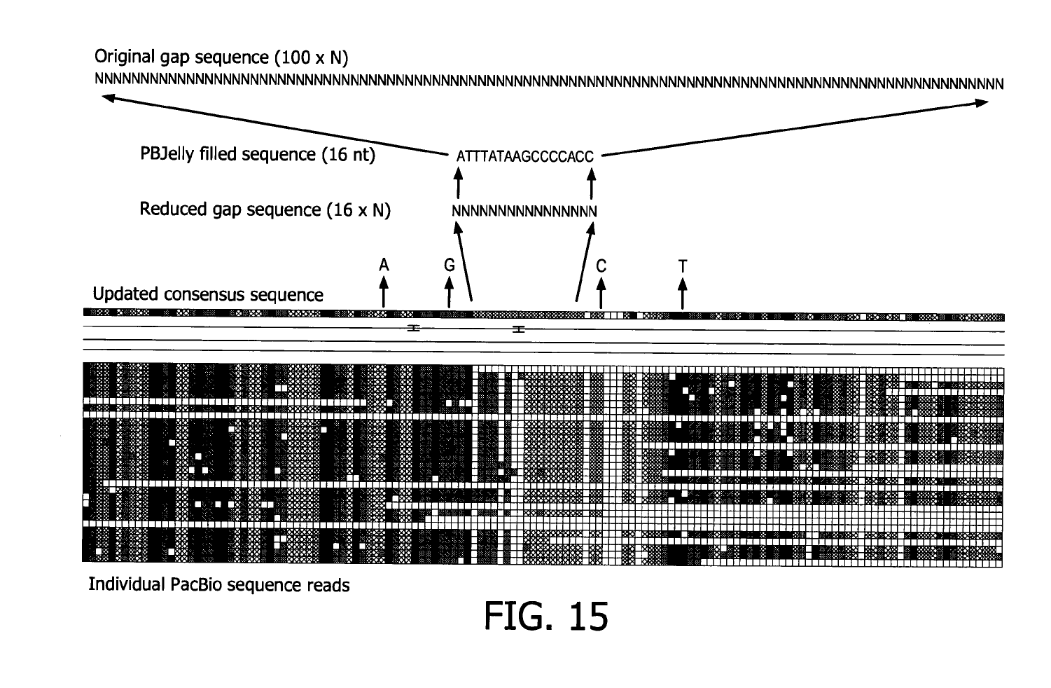

FIG. 1A: Schematic representation of Single sample—Single KNSS—Single Restriction enzyme—Single adaptors.

Analogous to FIG. 1 for one KNSS only. Only the fragment that has the KNSS on one end and the adaptor at the other end is capable of annealing to the circularization probe, followed by ligation and sequencing. Other fragments do not anneal to the circularization probe, or, if they do cannot be ligated to form a circular structure that can be sequenced.

FIG. 2: Single sample—Single KNSS—Single Restriction enzyme—Single adaptors—NO spacer sequence

Single KNSS sequence detection using an adaptor that ligates to the bottom strand of the restriction fragment. DNA is digested using a restriction enzyme (EcoRI). An adaptor is ligated and the ligation products are denatured. The denatured products are circularized using an oligonucleotide that is homologous to the adaptor sequence and the Known Nucleotide Sequence Section sequence. The ends of the circularized and denatured products are ligated. The generated ligated products are sequenced with which the Known Nucleotide Sequence Section sequence and flanking sequence information is determined.

FIG. 3: Single sample—Multiple KNSS—Single Restriction enzyme—Single adaptors—NO spacer sequence

Multiple KNSS sequence detection using a single adaptor. DNA is digested using a restriction enzyme (EcoRI). An adaptor is ligated and the ligation products are denatured. A subset of the denatured products are circularized using oligonucleotides homologous to the adaptor sequence and the Known Nucleotide Sequence Section sequences. The ends of the circularized and denatured products are ligated and subsequently sequenced.

FIG. 4: Multiple samples—Single KNSS—Single Restriction enzyme—Multiple adaptors (including sample ID)—NO spacer sequence.

Single KNSS sequence detection in two samples using an adaptor containing an identifier sequence. DNA of two samples is digested using a restriction enzyme. A sample specific adaptor is ligated and the ligation products are denatured. A subset of the denatured products are circularized using oligonucleotides homologues to the adaptor sequence and the Known Nucleotide Sequence Section sequence. The ends of the circularized and denatured products are ligated and subsequently sequenced.

FIG. 5: Single sample—Multiple KNSS—Single Restriction enzyme—Single adaptor—Single spacer sequence

Multiple KNSS sequence detection in a single sample using a single adaptor: DNA is digested using a restriction enzyme. An adaptor is ligated and the ligation products are denatured. A subset of the denatured products is circularized using oligonucleotides homologous to the adaptor sequence and the KNSS. The circularization oligonucleotides are partially double stranded and introduce a spacer sequence. The ends are ligated and subsequently the targeted fragments sequenced.

FIG. 6: Single sample—Multiple KNSS—Single Restriction enzyme—Single adaptor—Multiple spacer sequences

Multiple KNSS sequence detection in a single sample:

DNA is digested using a restriction enzyme. An adaptor is ligated and the ligation products are denatured. A subset of the denatured products is circularized using oligonucleotides homologous to the adaptor sequence and the KNSS. The circularization oligonucleotides are partially double stranded and introduce target specific spacer sequences. The ends are ligated and subsequently the targeted fragments sequenced.

FIG. 7: Single sample—Single Known Nucleotide Sequence Section—random fragmentation—Single adapter—NO spacer sequence

Single Known Nucleotide Sequence Section sequence detection using an adapter that ligates to the top strand of the fragment: DNA is randomly fragmented. An adapter is ligated and the ligation products are denatured. The denatured products are circularized using an oligonucleotide that is homologues to the adapter sequence and the Known Nucleotide Sequence Section sequence, which might be situated internal of the fragment. The (optionally) non hybridizing end of the fragment (flap) is removed and the resulting ends are ligated. The generated ligated products are sequenced with which the Known Nucleotide Sequence Section sequence and flanking sequence information is determined.

FIG. 8: Single sample—Single Known Nucleotide Sequence Section—random fragmentation—Single adapter—NO spacer sequence

Single Known Nucleotide Sequence Section sequence detection using an adapter that ligates to the bottom strand of the fragment: DNA is randomly fragmented. An adapter is ligated and the ligation products are denatured. The denatured products are circularized using an oligonucleotide that is homologues to the adapter sequence and the Known Nucleotide Sequence Section sequence, which might be situated internal of the fragment. The (optionally) non hybridizing end of the fragment is removed and the resulting ends are ligated. The generated ligated products are sequenced with which the Known Nucleotide Sequence Section sequence and flanking sequence information is determined.

FIG. 9: Single sample—Multiple Known Nucleotide Sequence Sections—random fragmentation—Single adapter—NO spacer sequence

Multiple Known Nucleotide Sequence Section sequence detection using a single adapter: DNA is randomly fragmented. An adapter is ligated and the ligation products are denatured. A subset of the denatured products are circularized using oligos homologues to the adapter sequence and the Known Nucleotide Sequence Section sequences which might be situated internal of the fragment. The (optionally) non hybridizing ends of the fragments are removed and the resulting ends are ligated. The generated ligated products are sequenced with which the Known Nucleotide Sequence Section sequences and their flanking sequence information is determined.

FIG. 10: Multiple samples—Single Known Nucleotide Sequence Section—random fragmentation—Multiple adapters (including sample ID)—NO spacer sequence

Single Known Nucleotide Sequence Section sequence detection in two samples using an adapter containing an identifier sequence: DNA of two samples is randomly fragmented. A sample specific adapter is ligated and the ligation products are denatured. A subset of the denatured products are circularized using oligos homologues to the adapter sequence and the Known Nucleotide Sequence Section sequence which might be situated internal of the fragment. The (optionally) non hybridizing ends of the fragments are removed and the resulting ends are ligated. The generated ligated products are sequenced with which the Known Nucleotide Sequence Section sequences and their flanking sequence information is determined.

FIG. 11: Single sample—Multiple Known Nucleotide Sequence Sections—random fragmentation—Single adapter—Single spacer sequence

Multiple Known Nucleotide Sequence Section sequence detection in a single sample using a single adapter:

DNA is randomly fragmented. An adapter is ligated and the ligation products are denatured. A subset of the denatured products are circularized using oligos homologues to the adapter sequence and the Known Nucleotide Sequence Section sequences which might be situated internal of the fragment. The circularization oligos are partially double stranded and introduce a spacer sequence. The (optionally) non hybridizing ends of the fragments are removed and the resulting ends are ligated. The generated ligated products are sequenced with which the Known Nucleotide Sequence Section sequences and their flanking sequence information is determined.

FIG. 12: Single sample—Multiple Known Nucleotide Sequence Sections—random fragmentation—Single adapter—Single spacer sequence

Multiple Known Nucleotide Sequence Section sequence detection in a single sample using a single adapter: DNA is randomly fragmented. An adapter is ligated and the ligation products are denatured. A subset of the denatured products are circularized using oligos homologues to the adapter sequence and the Known Nucleotide Sequence Section sequences which might be situated internal of the fragment. The circularization oligos are partially double stranded and introduce a Known Nucleotide Sequence Section specific spacer sequence. The (optionally) non hybridizing ends of the fragments are removed and the resulting ends are ligated. The generated ligated products are sequenced with which the Known Nucleotide Sequence Section sequences and their flanking sequence information is determined.

FIG. 13: Fragment length analysis after DNA repair, dA-tailing and adapter ligation.

FIG. 14: Agilent Bioanalyzer result of purified amplified targeted circularized products. On the horizontal axis migration time is depicted, which is indicative for the fragment lengths. The vertical axis indicates the fluorescent intensity, which is a measure for the concentration of a fragment.

FIG. 15: Alignment of 26 individual PacBio sequence reads (below) to the updated reference sequence. The updated reference sequence contains (artificially) inserted 16 N nucleotides for purposes of this example. Output of the PBJelly software contains the indicated filled sequence of 16 nt.

### EXAMPLES

### Example 1

Targeted sequencing Using Sequence Tags

Protocol

The approach contained the following steps:

1 Restriction Ligation (RL) of Qenomic DNA

An EcoRI restriction was performed on 500 ng DNA material and a modified EcoRI adaptor was ligated on the 3′ ends of the EcoRI fragments. EcoRI was used, as the tags from the physical map used were generated with EcoRI. However, in principle any restriction enzyme can be used.

2 Circularization and Ligation Using a Pool of Taq Sequences

A mixture was made of 37 biotinylated primers containing 13 nucleotides complementing the EcoRI adaptor and 18 nucleotides complementing the tag sequence (circularization probe mix). Circularization reactions were assembled, denatured for 10 minutes at 95° C. and cooled down to 75° C. Ligation mix containing thermo stabile ligase was added and the temperature was lowered overnight to 45° C. creating a complex of biotinylated circularization probe with circular ligated specific tag-EcoRI fragments. (circularization complex)

3 Capturing

The circularized complexes were bound to Dynabeads M-270 Streptavidin beads by means of the biotine group present in the circularization probes. The supernatant was removed, the beads were washed and the wash buffer was removed. The bound circulated fragments separated from the circularization probes using a heat treatment (5 min at 95° C.) in 20 μl Tris EDTA (TE).

4 Exo-Nuclease Treatment

On 10 μl captured fragments an exo-nuclease treatment was performed to degrade remaining linear (=non circular) fragments.

5. Enrichment

A standard rolling circle Templiphy reaction was performed on the captured fragment and on the exonuclease treated captured fragments. Positive products were seen for the captured fragments and for the exo nuclease treated captured fragments on 1% agarose gel.

6 Quantification

Q-PCR was performed on:

10 times diluted Templiphy captured fragments

10 times diluted Templiphy Exonuclease treated captured fragments

7. Results Summary

To check the quality of the RL reactions (=step 1), amplifications were performed using primers designed on the sequence tags in combination with a primers based on the adaptor sequence that was used in the RL reaction. This resulted in products ranging in sizes from 500-3500 bp after visualization on a 1% agarose gel. The enrichment amplification in step 5 resulted in products in the enriched samples. Q-PCR results showed that there was a clear difference in Cp values in the enriched samples, when compared to the non-enriched controls. Calculated enrichment was 1K-32K times. Duplo sample results were within 2Cp values. Mapping of the generated sequences showed that many reads were mapped across the genome, however there were scaffolds that contained significantly more reads and higher coverage than others.

### Example 2

Targeted Gap Filling in Maize

Protocol

The approach contained the following steps:

1 Fragmentation of Genomic DNA

500 ng genomic DNA material was fragmented to ˜10 Kbp using g-TUBE™ (Covaris®) fragmentation. The DNA ends were repaired (blunted) and a 3′ A nucleotide was added (=dA tailing). A modified adaptor was ligated to the 3′ ends of the fragments.

2 Circularization and Ligation Using a Pool of Tag Sequences

A mixture was made of 119 biotinylated oligonucleotides containing 18 nucleotides complementing the adaptor and (on average) 17 (range=13-23) nucleotides complementing the known sequence flanking the gap with unknown sequence in the selected genomic sequence region (circularization probe mix). Circularization reactions were assembled denatured for 10 minutes at 95° C. and lowered to 45° C. overnight. Ligation mix containing thermo stabile ligase and a DNA polymerase (having 3′-5′ exonuclease activity but lacking strand displacement activity and lacking 5′-3′ exonuclease activity) was added and the reaction mixture was incubated at 37° C. for 2 hrs with subsequently an increase of the temperature to 60° C. and an incubation of 30 minutes at 60° C. This created a complex of biotinylated circularization probe with specific ligated circularized fragments (circularization complex).

3 Capturing

The circularization complexes were bound to Dynabeads M-270 Streptavidin beads by means of the biotin group present in the circularization probes. The supernatant was removed, the beads were washed and the wash buffer was removed. The bound circularized fragments separated from the circularization probes using a heat treatment (5 min at 95° C.) in 20 μl Tris EDTA (TE).

4 Exo-Nuclease Treatment

On 100 μl of captured fragments an exo-nuclease treatment was performed using 40 μl SapExo mixture to degrade remaining linear (=non circular) fragments using an incubation of 15 minutes at 37° C. and 15 minutes at 80° C.

5. Amplification

A standard Genomiphy (=strand displacement) amplification reaction was performed on the exo nuclease treated captured fragments. In order to remove fragments with lengths below 3 Kbp an Ampure purification was performed.

6 PacBio Library Preparation

Library preparation for PacBio sequencing was performed according to the manufacturer's specifications, using blunt ended adapter ligation.

7 PacBio Sequencing

PacBio sequencing was performed according to the manufacturer's specifications using MagBead loading and a 3 hour movie time.

Results Summary

B73 maize DNA (5 μg) was fragmented to ˜10 Kbp fragments using g-TUBE shearing (Covaris) according the manufacturer's specifications, i.e. 6000 rpm for 60 seconds. Fragments smaller than 1.5 Kbp were removed using AMPure purification. Remaining fragments were end repaired using the NEBNext End Repair kit with manufacturer's specifications, after which purification was performed using AMPure beads. Subsequently A-tailing was performed using the NEBNext dA-tailing kit which involves incubating the DNA fragments with dATP and Klenow 3′-5′ Exo-DNA polymerase. Purification was performed using AMPure beads. Adapters containing a T overhang were ligated to the end repaired and A-tailed fragments. The adapter ligated fragments were purified using AMPure beads. Fragment size distribution was determined through analysis on the Agilent Tapestation. Results are shown in FIG. 13.

Circularization is initiated through an incubation of the adapter ligated fragments in combination with 119 circularization oligonucleotides which contain a complementary sequence to the adapter and a sequence complementary to the target region. Additionally the circularization oligonucleotides contain a biotin modification. Adapter ligated DNA is denatured at 95° C. for 10 minutes in the presence of a mix of the circularization oligo's. Subsequently the temperature is lowered from 75° C. to 45° C. and kept at 45° C. overnight. After circularization 3′ non matching parts of the DNA fragments are removed through incubation with T4-DNA polymerase and Taq DNA ligase in which the polymerase removes the non-matching DNA ends, if needed performs strand fill in, after which the ligase connects the now adjacent fragment ends and thus creates a circularized DNA fragment. DNA fragments with hybridized circularization oligonucleotides are isolated using streptavidin coated magnetic beads. To lower a-specific hybridization, the beads with coupled fragments are washed multiple times. Coupled fragments are eluted from the beads through incubation at 95° C. for 5 minutes. As the isolated DNA may contain non-circular molecules, linear fragments are removed through incubation with a mixture of Shrimp Alkaline Phophatase and an Exonuclease for 15 minutes at 37° C. The enzymes are inactivated at 80° C. for 10 minutes. Amplification of the remaining DNA is performed using the Genomiphy kit. Amplification products are purified using AMPure beads. Total yield was 3.5 ug. Length distribution was analyzed using the Agilent BioAnalyzer. Result is shown in FIG. 14. The products shown in FIG. 14 are used to prepare a PacBio sequencing library, which involved polishing the DNA and ligation of the SMRT bell adapter. Sequencing is performed using the manufacturer's specifications with MagBead loading and a 3 hour movie time. Sequencing yielded, after initial filtering, a total of 25,988 reads containing a total of 142,229,422 nucleotides, i.e. average read length was 5,472 nucleotides. The generated reads were screened for presence of the adapter sequence added early in the protocol and for the PacBio SMRT bell adapter sequence. If either adapter sequence was present, the corresponding read was split and the adapter sequence was removed. The resulting reads were used as input for the software tool PBJelly, which is able to close gaps in reference sequences. The steps in PBJelly involve mapping of the reads against the reference sequence of the 1 Mbp target region, determining if there are nucleotides mapped in the gaps. If so, the consensus sequence is determined and the reference sequence is updated. For visualization purposes, results from PBJelly were extracted and imported in the software package Tablet. An example of a filled gap is shown in FIG. 15. It shows that a gap of 100 unknown nucleotides is reduced and filled with 16 known nucleotides.

### CITATION LIST

### Patent Literature

WO200511236
WO2012003374
WO2011067378
WO2008153492
EP534858
WO2008007951
WO2010082815A1
WO2011074960A1
WO 03/004690
WO 03/054142
WO 2004/069849
WO 2004/070005
WO 2004/070007
WO 2005/003375
U.S. Pat. No. 6,045,994

### Non Patent Literature

Seo et al. (2004) Proc. Natl. Acad. Sci. USA 101:5488-93.
Quail et al., BMC Genomics 2012, 13:341
“Next Generation Genome sequencing”, M. Janitz Ed. (Wiley-Blackwell, 2008).
Marmur and Lane, Proc. Natl. Acad. Sci. USA 46:453 (1960)
Doty et al., Proc. Natl. Acad. Sci. USA 46:461 (1960)
Sambrook et al. (1989)
Sambrook and Russell (2001).
Vos et al 1995. AFLP: a new technique for DNA fingerprinting. Nucleic Acids Research 23(21): 4407-4414

1. Method for obtaining sequence information from a nucleic acid sample, the method comprising the steps of:
a) providing a nucleic acid sample wherein at least part of the nucleotide sequence information for the nucleic acid sample is available in the form of at least one Known Nucleotide Sequence Section;
b) fragmenting the nucleic acid sample to obtain one or more fragments;
c) optionally, blunting the ends of the fragments(s);
d) optionally, adding one or more 3′ nucleotides to the fragments;
e) ligating one or more adaptor(s) to one or both of the ends of the fragment(s) to obtain (an) adaptor-ligated fragment(s);
f) denaturing the adaptor-ligated fragment(s) to obtain (a) denatured adaptor-ligated fragment(s);
g) providing for at least one, preferably for each, optionally selected, Known Nucleotide Sequence Section-containing, denatured adaptor-ligated fragment a circularization probe that comprises at least part of the Known Nucleotide Sequence Section and at least part of the sequence of the adaptor;
h) combining the denatured adaptor-ligated fragment(s) with the circularization probe(s);
i) allowing the circularization probe(s) and the denatured adaptor-ligated fragment(s) to hybridize and form (a) circularized denatured adaptor-ligated fragment(s);
j) optionally, removing an overhang;
k) optionally, filling in missing nucleotides between (part of) the Known Nucleotide Sequence Section and (part of) the adaptor;
l) ligating the ends of the circularized adaptor-ligated fragment(s) to obtain (a) ligated circularized adaptor-ligated fragment(s); and
m) sequencing the ligated circularized adaptor-ligated fragment(s); wherein, for each fragment, sequence information of only one single Known Nucleotide Sequence section is required to obtain sequence information of the ligated circularized adaptor-ligated fragment(s).

2. Method for obtaining sequence information from a nucleic acid sample, the method comprising the steps of:
a) providing a nucleic acid sample wherein at least part of the nucleotide sequence information for the nucleic acid sample is available in the form of at least one Known Nucleotide Sequence Section;
b) fragmenting the nucleic acid sample to obtain one or more fragments;
c) optionally, blunting the ends of the fragments(s);
d) optionally, adding one or more 3′ nucleotides to the fragments;
e) ligating one or more adaptor(s) to one or both of the ends of the fragment(s) to obtain (an) adaptor-ligated fragment(s);
f) providing for at least one, preferably for each, optionally selected Known Nucleotide Sequence Section-containing, adaptor-ligated fragment a circularization probe that comprises at least part of the Known Nucleotide Sequence Section and at least part of the sequence of the adaptor;
g) combining the adaptor-ligated fragment(s) with the circularization probe(s);
h) denaturing the adaptor-ligated fragment(s) to obtain (a) denatured adaptor-ligated fragment(s);
i) allowing the circularization probe(s) and the denatured adaptor-ligated fragment(s) to hybridize and form (a) circularized denatured adaptor-ligated fragment(s);
j) optionally, removing an overhang;
k) optionally, filling in missing nucleotides between (part of) the Known Nucleotide Sequence Section and (part of) the adaptor;
l) ligating the ends of the circularized adaptor-ligated fragment(s) to obtain (a) ligated circularized adaptor-ligated fragment(s); and
m) sequencing the ligated circularized adaptor-ligated fragment(s); wherein, for each fragment, sequence information of only one single Known Nucleotide Sequence section is required to obtain sequence information of the ligated circularized adaptor-ligated fragment(s).

3. Method for obtaining sequence information from a nucleic acid sample, the method comprising the steps of:
a) providing a nucleic acid sample wherein at least part of the nucleotide sequence information for the nucleic acid sample is available in the form of at least one Known Nucleotide Sequence Section
b) fragmenting the nucleic acid sample to obtain one or more fragments;
c) optionally, blunting the ends of the fragments(s);
d) adding one or more 3′ nucleotides, preferably 10 to 20 nucleotides to the fragment(s) to obtain (a) nucleotide-elongated fragment(s);
e) denaturing the nucleotide-elongated fragment(s) to obtain (a) denatured nucleotide-elongated fragment(s);
f) providing for at least one, preferably for each, optionally selected Known Nucleotide Sequence Section-containing, denatured nucleotide-elongated fragment a circularization probe that comprises at least part of the Known Nucleotide Sequence Section and at least part of the sequence of the nucleotide-elongated sequence;
g) combining the denatured nucleotide-elongated fragment(s) with the circularization probe(s);
h) allowing the circularization probe(s) and the denatured nucleotide-elongated fragment(s) to hybridize and form (a) circularized denatured nucleotide-elongated fragment(s);
i) optionally, removing an overhang;
j) optionally, filling in missing nucleotides between (part of) the Known Nucleotide Sequence Section and (part of) the nucleotide-elongated sequence;
k) ligating the ends of the circularized adaptor-ligated fragment(s) to obtain (a) ligated circularized nucleotide-elongated fragment(s); and
l) sequencing the ligated circularized nucleotide-elongated fragment(s); wherein, for each fragment, sequence information of only one single Known Nucleotide Sequence section is required to obtain sequence information of the ligated circularized nucleotide-elongated fragment(s).

4. The method according to any one of claims 1-3, wherein the removal of the overhang is by means of an enzyme.

5. The method according to claim 4, wherein the enzyme is an endonuclease.

6. The method according to claim 5 wherein the endonuclease is a flap endonuclease

7. The method according to claim 4, wherein the enzyme is a polymerase with nuclease activity.

8. The method according to any one of the previous claims, wherein the fragments are provided by random fragmentation, preferable selected form the group consisting of shearing, sonication or nebulization.

9. The method according to any one of the previous claims, wherein fragmenting the nucleic acid is by digesting with one or more nuclease enzymes.

10. The method according to claim 9, wherein the nuclease enzyme is a restriction endonuclease enzyme.

11. The method according to claim 10, wherein a restriction enzyme digestion site is comprised in the Known Nucleotide Sequence Section.

12. The method according to claim 10, wherein a restriction enzyme digestion site is located outside the Known Nucleotide Sequence Section.

13. The method according to claim 10, wherein the fragments are provided by digesting the nucleic acid sample with one or more restriction endonuclease enzyme(s) wherein the restriction enzyme(s) digest(s) at the restriction enzyme digestion site(s) to obtain (a) restriction-enzyme digested fragment(s).

14. The method according to claim 13, wherein the restriction endonuclease enzyme digestion site(s) and the restriction endonuclease enzyme recognition site(s) are located at the same position (Class II restriction endonuclease).

15. The method according to claim 13, wherein the restriction endonuclease enzyme digestion site(s) and the restriction endonuclease enzyme recognition site(s) are not located at the same position (Class IIS or IIB restriction endonuclease)

16. The method according to claim 13, wherein the restriction endonuclease enzyme digestion site(s) is located outside the restriction endonuclease enzyme recognition side on one side (Class IIS restriction endonuclease) or on both sides (Class IIB restriction endonuclease).

17. The method according to any one of the previous claims, wherein the Known Nucleotide Sequence Section is located at one of the ends of the fragment.

18. The method according to any one of the previous claims, wherein the Known Nucleotide Sequence Section is located at a position removed from the ends of the fragments, preferably at a position at least 5, 10, 15, 20, 30, 50, 75 or 100 nucleotides from (one of) the ends of the fragment.

19. The method according to any one of the previous claims, wherein the adaptor is a double stranded adaptor.

20. The method according to any one of the previous claims, wherein the adaptor is a single stranded adaptor.

21. The method according to claim 1 or 2, wherein the fragmentation and the ligation of the adaptor are performed simultaneously.

22. The method according to any one of claims 1-3, wherein hybridizing the adaptor-ligated or nucleotide-elongated fragment with the circularization probe results in the creation of an overhang in the circularized denatured adaptor-ligated or nucleotide-elongated fragment(s).

23. The method according to any one of the previous claims, wherein at least part of the nucleotide sequence information of the nucleic acid is known in the form of a plurality of Known Nucleotide Sequence Sections that optionally comprise a restriction enzyme digestion site.

24. The method to any one of the previous claims, wherein a plurality of samples containing each one or more Known Nucleotide Sequence Sections are analysed to thereby obtain further sequence information.

25. The method according to any one of the previous claims, wherein the circularization probe comprises a spacer sequence.

26. The method according to claim 25, wherein the spacer is located between the part of the Known Nucleotide Sequence Section, and the part of the sequence of the at least one adaptor or the nucleotide-elongated sequence.

27. The method according to claim 25-26, wherein before ligation the gap caused by the spacer sequence is filled in, preferably by a polymerase reaction.

28. The method according to claim 25-27, wherein the spacer sequence is double-stranded.

29. The method according to claim 25-27, wherein the spacer sequence is single-stranded.

30. The method according to claims 25-29, wherein the spacer sequence comprises an identifier sequence.

31. The method according to claim 30, wherein the identifier sequence is a sample-specific identifier.

32. The method according to claims 30-31, wherein the identifier sequence is a Known Nucleotide Sequence Section-specific identifier.

33. The method according to claims 25-32, wherein the spacer sequence comprises at least one primer sequence.

34. The method according to claim 33, wherein the primer sequence is an amplification primer sequence and/or a sequencing primer sequence.

35. The method according to claim 34, wherein the amplification and sequencing primer are combined in a combined amplification/sequencing primer.

36. The method according to any one of the previous claims, wherein the denaturation of the adaptor-ligated or nucleotide-elongated fragment(s) and the combination of the denatured adaptor-ligated or nucleotide-elongated fragment(s) with the circularization probe(s) are performed in reverse order.

37. The method according to any one of the previous claims wherein the fragmenting or digesting step and the ligation step are performed simultaneously.

38. The method according to any one of the previous claims, wherein the one or more adaptor(s) or nucleotide-elongated sequence comprises (an) identifier sequence(s).

39. The method according to claim 38, wherein the identifier sequence is a sample-specific identifier.

40. The method according to claim 38-39 wherein the identifier sequence is a Known Nucleotide Sequence Section-specific identifier.

41. The method according to any of the previous claims, wherein the one or more adaptor(s) or nucleotide-elongated sequence comprise(s) at least one primer sequence.

42. The method according to claim 41, wherein the primer sequence is an amplification primer sequence and/or a sequencing primer sequence.

43. The method according to claims 41-42, wherein the amplification and sequencing primer are combined in a combined amplification/sequencing primer.

44. The method according to any one of the previous claims, wherein after fragmentation, the fragments are pooled.

45. The method according to any one of the previous claims, wherein the adaptor-ligated or nucleotide-elongated fragments are pooled after the adaptor-ligation or nucleotide elongation step and before the sequencing step.

46. The method according to any one of the previous claims, wherein after the ligation step, the ligated circularized adaptor-ligated fragment(s) or ligated circularized nucleotide-elongated fragment(s) are amplified by using at least one random primer.

47. The method according to claim 46, wherein amplification is performed using a polymerase having strand-displacement activity, such as phi29.

48. The method according to any one of the previous claims, wherein after the ligation step, the ligated circularized adaptor-ligated fragment(s) or ligated circularized nucleotide-elongated fragment(s) are amplified by using at least one primer that can anneal to at least part of the sequence of the at least one Known Nucleotide Sequence Section, or to at least part of the sequence of the adaptor or nucleotide-elongated sequence, or to both.

49. The method according to claim 48, wherein the amplification is rolling circle amplification reaction.

50. The method according to claim 46-49, wherein the at least one primer comprises an identifier sequence.

51. The method according to claim 50, wherein the identifier sequence is specific for the sample and/or Known Nucleotide Sequence Section.

52. The method according to any one of the previous claims, wherein the identifier sequence does not contain two or more identical consecutive bases and/or wherein the identifier sequences mutually all differ by at least two bases.

53. The method according to any one of the previous claims, wherein the ligated circularized adaptor-ligated fragment(s) or ligated circularized nucleotide-elongated fragment(s) are further fragmented before the sequencing step.

54. The method according to claim 53, wherein the further fragmentation is achieved by shearing, nebulization, sonication, restriction enzyme digestion, and/or nuclease treatment.

55. The method according to claim 54, wherein the further fragmentation by restriction enzyme digestion is with a restriction endonuclease enzyme that has a different recognition sequence or cutting site than the restriction endonuclease enzyme of claim 10.

56. The method according to any of the previous claims where a plurality of Known Nucleotide Sequence Section and/or a multiplicity of samples is used.

57. The method according to any one of the previous claims, wherein after the step wherein the circularized adaptor-ligated fragment(s) or circularized nucleotide-elongated fragment(s) is ligated, an exo-nuclease treatment is performed.

58. The method according to any one of the previous claims, wherein the at least one circularization probe is provided with an affinity moiety or probe such as biotin, or wherein the primer in the amplification step contains an affinity moiety or probe such as biotin.

59. The method according to any one of the previous claims, wherein the circularized, adaptor-ligated fragment(s) or circularized nucleotide elongated fragments are captured after addition of the circularization probe.

60. The method according to claim 58 or 59, wherein the, optionally amplified and/or ligated) circularized adaptor-ligated fragment(s) or circularized nucleotide elongated fragment(s) are captured using the affinity moiety on the primer or the circularization probe.

61. The method according to any one of the previous claims, wherein ligation of the one or more adaptor(s) in the adaptor ligation step occurs at the 3′ end of the fragmented, optionally restriction enzyme digested, fragments(s).

62. The method according to any one of the previous claims, wherein ligation of the one or more adaptor(s) in the adaptor ligation step occurs at the 5′ end of the fragmented, optionally restriction enzyme digested, fragments(s).

63. The method according to any one of the previous claims, wherein the method is used for re-sequencing a nucleic acid.

64. The method according to any one of the previous claims, wherein the method is used for determining sequence variation in the vicinity of the Known Nucleotide Sequence Section.

65. The method according to any one of the previous claims, wherein the method is used for gap closure in genome sequences at one or more positions where at least one Known Nucleotide Sequence Section is available.

66. The method according to any one of the previous claims, wherein the further sequence information is linked to existing sequence information such as from a physical map or draft genome sequence.

67. The method according to any one of the previous claims, wherein the at least one Known Nucleotide Sequence Section is linked to a region in which a trait or gene is located.

68. The method according to claim 47, wherein the trait or gene is a plant trait or gene.

69. Use of a ligated, circularized adaptor-ligated fragment, or ligated, circularized nucleotide-elongated fragment wherein at least part of the fragment comprises a Known Nucleotide Sequence Section for obtaining sequence information.

70. Use according to claim 69, wherein the fragment is provided by random fragmentation (shearing, sonication, nebulization) of a nucleic acid sample.

71. Use according to claim 69, wherein the fragment is provided by restriction enzyme digestion of a nucleic acid sample.

72. Use according to claim 71, wherein a restriction enzyme digestion site is comprised in the Known Nucleotide Sequence Section.

73. Use according to claim 71, wherein a restriction enzyme digestion site is located outside the Known Nucleotide Sequence Section.
